(19) United States
(12) Patent Application Publication
(10) Pub. No.: US 2012/0258944 A1
(43) Pub. Date: Oct. 11, 2012

(54) ANTIOXIDANTS FOR USE IN THERAPY

(75) Inventors: Lars Engman, Enebyberg (SE); Henrik Alexander Johansson, Uppsala (SE)

(73) Assignee: KAROLINSKA INSTITUTET INNOVATIONS AB, Solna (SE)

(21) Appl. No.: 13/499,872

(22) PCT Filed: Oct. 4, 2010
(86) PCT No.: PCT/EP2010/064767

§ 371 Date: Jun. 21, 2012

Related U.S. Application Data
(60) Provisional application No. 61/248,332, filed on Oct. 2, 2009.

(51) Int. Cl. A61K 31/33; A61P 17/18

(52) U.S. Cl. 514/183

(57) ABSTRACT

Compounds based on the following structures: wherein each of R1, R2, R3 and R4 is independently selected from: hydrogen, alkyl, and substituted alkyl; and each of R5, R6 and R7 is independently selected from: hydrogen, alkyl, substituted alkyl, alkoxy, substituted alkoxy, N,N-dialkylamino, substituted N,N-dialkylamino, N-monoalkylamino, substituted N-monoalkylamino, and electron-donating substituents are useful in the treatment of disorders or conditions caused by or involving free radical-mediated or oxidative tissue damage.

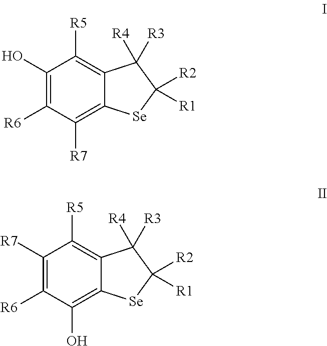

The present invention relates to selenium-containing compounds for use as medicaments, and in particular to compounds comprising a phenol fused to a five-membered selenium-containing ring, for use in the treatment of disorders or conditions caused by or involving free radical-mediated or oxidative tissue damage.

Antioxidants are useful in many fields, including medical fields where problems caused by or involving free radical-mediated or oxidative tissue damage need treatment.

Numerous potential antioxidants have been studied in the hope of finding some which can be therapeutically useful. Amongst the large number of publications in this field, some relate to selenium-containing compounds. The use of selenium-containing compounds is associated with several disadvantages. One disadvantage is that their toxicology can be difficult to predict, and in many cases they exhibit toxicity by various mechanisms as summarised in for example Chem. Rev. 2004, 104, 6255-6285.

Several investigations and publications have indicated that certain selenium-containing compounds are not effective as medically useful antioxidants, and in some cases have taught that tellurium-containing compounds are highly effective whereas corresponding selenium-containing compounds are not.

For example, J. Am. Chem. Soc. 2001, 123, 3434-3440, a publication by the present inventor and others, discloses various studies on the following range of compounds:

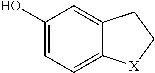

### X═O, S, Se or Te

This publication tries to assess the antioxidant character of these compounds by several tests and analyses.

Firstly, from a theoretical chemistry viewpoint, by considering redox properties of the compounds, the authors of J. Am. Chem. Soc. 2001, 123, 3434-3440 make postulations regarding bond dissociation enthalpies and oxidation potentials. However, because many other factors come into play, it is not reasonable to extrapolate such characteristics to predict how the compounds would behave as antioxidants in a real biological system.

Secondly, the extent to which the compounds inhibit the azo-initiated peroxidation of linoleic acid in a two-phase system is studied, and the publication states that this gives a reasonable indication of how the compounds inhibit lipid peroxidation. The publication indicates that, in the absence of thiol reducing agent, none of the compounds are as good as the natural antioxidant alpha-tocopherol at inhibiting peroxidation, and that the organoselenium compound (X═Se) is a poor inhibitor. In the presence of a reducing agent, according to this test method, the tellurium compound (X═Te) has the best antioxidant capacity.

Thirdly, the J. Am. Chem. Soc. 2001, 123, 3434-3440 publication teaches that hydroperoxide decomposition is perhaps the most important duty for preventive antioxidants, and that in biological systems this task is fulfilled by selenium-containing glutathione peroxidases and catalase. The publication refers to a coupled reductase method as providing a convenient test for assessing thiol peroxidase activity. It discloses that catalyst activity in the reaction between glutathione and hydroperoxide is insignificant for the organoselenium derivative (X═Se). In contrast, the organotellurium derivative (X═Te) is shown to be a highly active catalyst according this method.

Fourthly, the publication investigates the inhibition of lipid peroxidation in liver microsomes. Whereas the organotellurium derivative (X═Te) is stated to be a potent inhibitor, the other three analogues (X═O,S,Se) are not effective according to this method.

Another publication by the present inventor and others is J. Org. Chem. 2007, 72, 2583-2595. This uses similar test methods to those used in J. Am. Chem. Soc. 2001, 123, 3434-3440, including the two-phase method to assess the inhibition of peroxidation of linoleic acid. Although J. Org. Chem. 2007, 72, 2583-2595 studies variously substituted selenium-containing compounds wherein X is Se, in more detail, it reaches no clear conclusions. It finds that compounds often do not behave as predicted, and that there are several trends and aspects which are incompletely understood. Several selenium-containing compounds are disclosed as having poor regenerability.

Now, however, further work by the inventor has shown that organo-selenium compounds as defined below are surprisingly effective in biological systems, and in fact that some of the previous test results cannot be extrapolated to give a true indication of efficacy in vivo. Experiments as described below have compared compounds of the invention with various other compounds which fall outside the definition of the present invention (“comparative compounds”). The comparative compounds work better than compounds of the invention in the two-phase model. However, in biological systems, the compounds of the present invention work better than comparative compounds. This was completely unexpected to the present inventor.

It is surprising that the compounds of the present invention work better than comparative compounds, when in the two-phase model they perform less well than the comparative compounds. Furthermore, the inventor has shown that the compounds of the present invention possess the additional advantage of being pharmaceutically acceptable when so many other selenium-containing compounds are toxic.

From a first aspect, therefore, the present invention provides a compound comprising the following formula I, or a pharmaceutically acceptable salt thereof, for use in therapy:

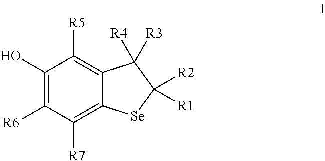

wherein
each of R1, R2, R3 and R4 is independently selected from:
hydrogen,
alkyl, and
substituted alkyl;
   and
   each of R5, R6 and R7 is independently selected from:
hydrogen,
alkyl,
substituted alkyl,
alkoxy,
substituted alkoxy,
N,N-dialkylamino,
substituted N,N-dialkylamino,
N-monoalkylamino,
substituted N-monoallylamino, and
electron-donating substituents.

From a second aspect, the present invention provides a compound comprising the following formula II, or a pharmaceutically acceptable salt thereof, for use in therapy:

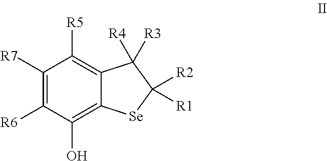

wherein
each of R1, R2, R3 and R4 is independently selected from:
hydrogen,
alkyl, and
substituted alkyl;
   and
   each of R5, R6 and R7 is independently selected from:
hydrogen,
alkyl,
substituted alkyl,
alkoxy,
substituted alkoxy,
N,N-dialkylamino,
substituted N,N-dialkylamino,
N-monoalkylamino,
substituted N-monoalkylamino, and
electron-donating substituents.

It will be seen that formula II differs from formula I only in that R7 and OH are exchanged. This means that, in both formula I and formula II, the selenium atom, R5 and R6 are all ortho- or para- to OH. Therefore, the two structural types are closely related in terms of electronic effects.

The compounds of formulae I and II are useful as medicaments in the treatment of disorders or conditions caused by or involving free radical-mediated or oxidative tissue damage.

The disorders or conditions include for example: ischemic or reperfusion injuries, thrombosis, embolism, neoplasms, cancer, Parkinson's disease, Alzheimer's disease, atherosclerosis, allergic/inflammatory conditions such as bronchitis, asthma, rheumatoid arthritis, ulcerative cholitis, Crohn's disease, cataract, respiratory distress syndrome, damage caused by chemicals, radiation, antineoplastic or immunosuppressive agents, ischemia/reperfusion injury in the heart, kidney and CSN and post-operative ischemia/reperfusion injury, organ preservation, burn injury, wound healing, and IBS (irritable bowel syndrome).

Preferred therapeutic applications are organ preservation, treatment of burn injury and treatment of rheumatoid arthritis.

Also covered by the present invention are pharmaceutical compositions comprising the compounds of the present invention and pharmaceutically acceptable diluents, excipients and/or carriers.

The core structures of formulae I and II are responsible for the enhanced activity of the compounds based on them. It is within the scope of the present invention to add substituents or moieties to the core structure, so long as the compounds still exhibit the therapeutic activity. For example, various groups are listed within the definitions of each of R1, R2, R3, R4, R5, R6 and R7 above, including some with optional substituents. The substituents may for example be: halogen (e.g. F, Cl, Br or I); OH, alkoxy, SH, NH₂, N-alkylamino, N,N-dialkylamino, COOH, CHO, NO₂, aryl or heteroaryl. The substituents may be further substituted by for example any of these, e.g. an alkoxy substituent may have a further substituent on its alkyl chain.

Alkyl as used herein preferably denotes C₁-C₁₅ alkyl. The alkyl may be saturated or unsaturated, unbranched or branched, or any combination thereof. The skilled person is well aware that different alkyl chain lengths affect the hydrophilicity/lipophilicity of compounds, and therefore in some cases short alkyl chain lengths are preferred whereas in other cases more “greasy” longer alkyl chains are preferred. This is an advantage of the present invention because it allows the tailoring of molecules.

The nature of the alkyl groups will now be discussed in more detail with reference to R1, R2, R3 and R4. Whilst all of these are shown in formulae I and II, in many of the preferred compounds some of R1, R2, R3 and R4 are hydrogen. In one preference, three of these are hydrogen and one of these is alkyl. In a further preference, two of these are hydrogen and two of these are alkyl (the same or different). It is within the scope of the present invention that all four may be hydrogen. It is also within the scope of the present invention that none, or one, may be hydrogen, and the remainder alkyl.

One or more of R1, R2, R3 and R4 may be alkyl groups such as C₁-C₁₅ alkyl. One preferred type of alkyl is lower alkyl (C₁-C₅ alkyl). A preferred alkyl group is methyl. In one preference R1 is methyl and R2, R3 and R4 are hydrogen. In another preference R3 is methyl and R1, R2, and R4 are hydrogen. In another preference each of R1, R2, R3 and R4 is hydrogen.

In a further preference, R1 is methyl and R2, R3, R4, R5, R6 and R7 are hydrogen. In another preference R3 is methyl and R1, R2, R4, R5, R6 and R7 are hydrogen. In another preference each of R1, R2, R3, R4, R5, R6 and R7 is hydrogen.

A further preferred alkyl group for R1, R2, R3 and/or R4 is ethyl.

Further preferences are the alteration of the above preferences by the addition of optional substituents on the alkyl groups; various possible substituents are listed above.

Thus it will be noted that the possibilities on the selenium-containing ring are defined in a focused way to cover those patterns which exhibit surprisingly effective results.

The possible substituents on the aromatic ring will now be discussed. Various possibilities for R5, R6 and R7 are defined above. It should be noted, firstly, that hydrogen may be present at one, two, or all of these positions, and that there is no need for other substituents in order for the compounds to be effective. However, some of the effective compounds do contain other moieties at one or more of these positions, and these moieties may optionally themselves carry further substituents.

With reference to R5, R6 and R7, the term “alkyl” has the same meaning as it does for R1, R2, R3 and R4. The term “alkoxy” is defined analogously, i.e. the same possible carbon chain lengths and other features and preferences apply as for “alkyl”. Similarly the same definitions, preferences and features apply, mutatis mutandis, to alkyl component(s) within alkylamino moieties. In particular, for example, a preferred alkyl part of these moieties is methyl.

It will also be noted that R5, R6 and R7 may be electron-donating substituents. Many of the moieties already listed for R5, R6 and R7 are of course inherently electron-donating substituents. This term also includes other substituents which are known to the skilled person as being electron-donating substituents, as well as those explicitly listed.

It is preferred for there to be electron-donating moieties at R5 and/or R6, in order to result in compounds which are particularly effective. Thus, R7 may be hydrogen whilst R5 and/or R6 is not hydrogen.

Thus the possibilities on the aromatic ring are defined in a focused way to cover those patterns which exhibit surprisingly effective results.

Some preferred compounds, and groups of compounds, of the invention are shown below. In these,
i) R10 is as defined for R1, preferably selected from C₁-C₅ alkyl;
ii) R30 is as defined for R3, preferably selected from C₁-C₅ alkyl;
iii) R50 is as defined for R5, preferably selected from H, C₁-C₅ alkyl, C₁-C₅ alkoxy, di-(C₁-C₅ alkyl)-amino or mono-(C₁-C₅ alkyl)amino; and
iv) R60 is as defined for R6, preferably selected from H, C₁-C₅ alkyl, C₁-C₅ alkoxy, di-(C₁-C₅ alkyl)-amino or mono-(C₁-C₅ alkyl)amino.

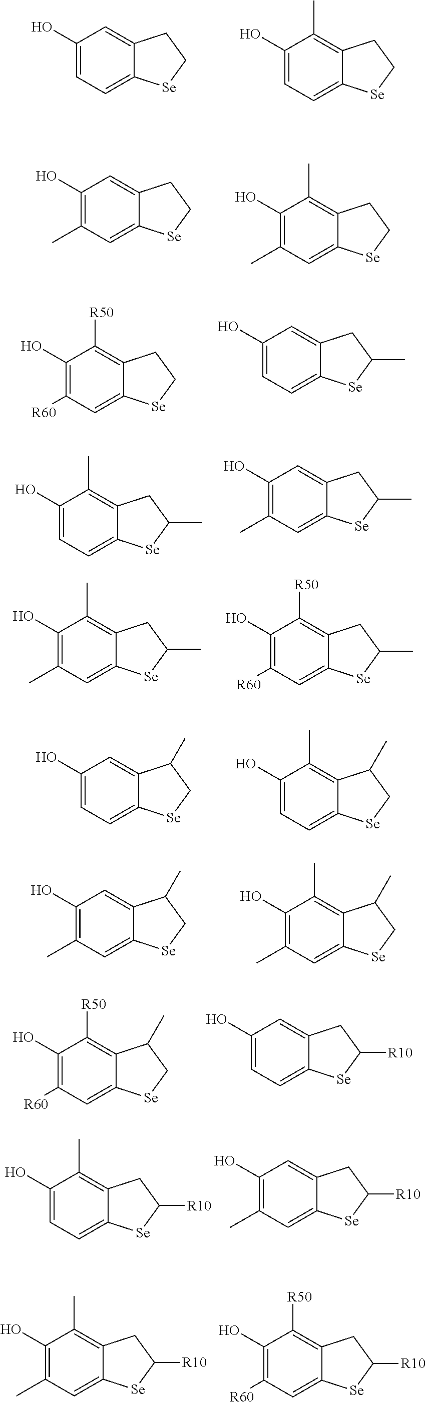

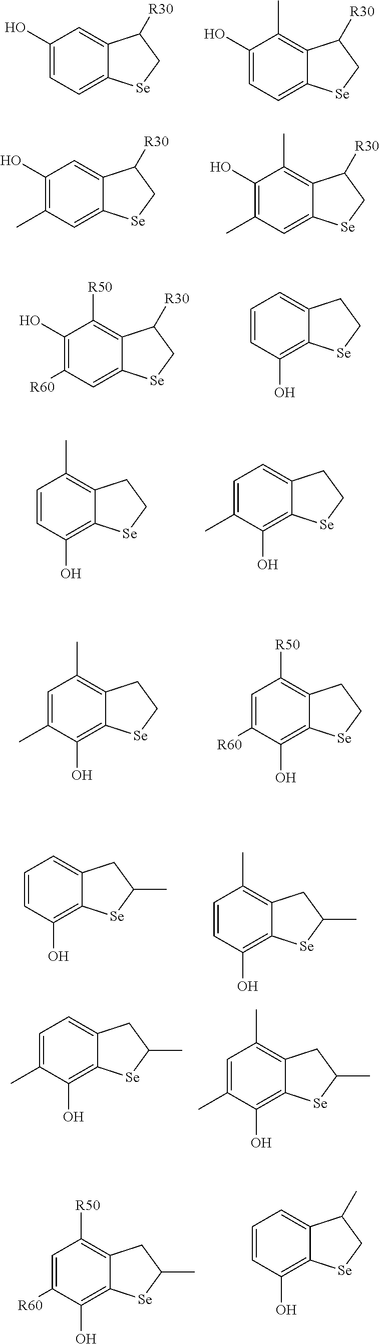

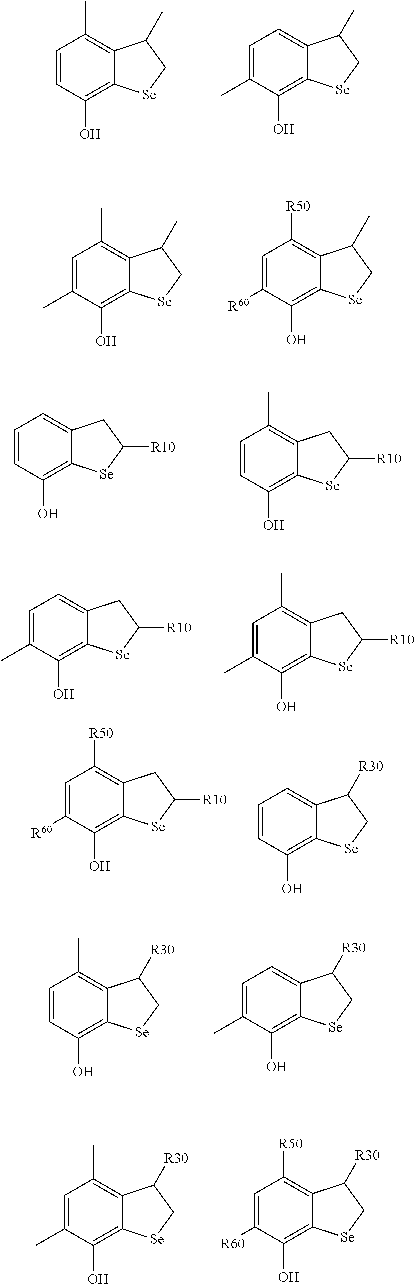

Compounds of formulae I and II may be made according to procedures known in the art, adapted where necessary within the ability of the skilled person. For example, preparative procedures for some of the compounds are given in J. Org. Chem. 2007, 72, 2583-2595 and J. Am. Chem. Soc. 2001, 123, 3434-3440.

The compound according to the present invention may optionally be used as a catalytic antioxidant. For example, it may be used under conditions which allow its regeneration, either because the system in which it is used inherently regenerates the compound and cycles the catalyst, or because an additional component is used to regenerate the catalyst.

Thus, the compound according to the present invention may also be combined with, or used in combination with, a reducing agent. The reducing agent may for example be a mild reducing agent. For example, suitable reducing agents include those which are suitable for regenerating the catalyst without causing undesirable side-effects. One suitable class of reducing agents is the class of thiols. Specific examples include N-acetylcysteine, cysteine, dithiothreitol, glutathione, ascorbic acid and sodium ascorbate.

In drug development, catalytic compounds are particularly advantageous since only small amounts are needed to achieve the pharmacological effects. The relatively low amounts needed significantly reduce the risks of toxic effects or other side effects, including those which may be associated with impurities in a final drug substance.

Additionally, catalytic use is economically and environmentally advantageous and minimizes the preparation and use of potentially hazardous materials. It allows the use of reducing agents which are low-cost, readily available and safe.

The compounds of the present invention can be used in a non-catalytic sense, but in that case they only have a finite effect, and for example can only destroy a limited number of peroxyl radicals before they are themselves converted to inactive compounds. It is therefore preferable for them to be regenerated by cheap, nontoxic reducing agents. It is advantageous that the compounds can act as chain-breaking antioxidants by catalyzing the decomposition of peroxyl radicals in the presence of mild reducing agents.

The present invention describes novel, regenerable compounds, and any acid or base addition salt or prodrug thereof, which are capable of acting as chain-breaking antioxidants by catalyzing the decomposition of peroxyl radicals in the presence of mild reducing agents. Compounds according to the present invention interfere with pathophysiologically important reactions in man and animals and thus effectively hamper the degradation of tissue constituent molecules as well as act to remove harmful products from such degradation. The compounds possess an ability to protect tissues against oxidative damage induced by overreacting host defence systems. Compounds according to the present invention are therefore useful for the pharmacological treatment of diseases in which free radical-mediated or oxidative tissue degradation occurs or where oxidants trigger pro-inflammatory receptors on cell surfaces. The reducing agents required for a catalytic mode of action of compounds according to formulae I and II could either be exogenous and present in the environment where the antioxidant effect is desired (for example glutathione or ascorbate) or they have to be supplemented together with compounds according to the present invention. Diseases such as inflammatory (including autoimmune inflammatory) conditions like asthma, bronchitis, various allergic skin and systemic disorders, Crohn's disease, ulcerative colitis, rheumatoid arthritis and other kinds of arthritis respond to such treatment. Compounds according to the present invention together with suitable reducing agents may also be used for intervention of cataract and the respiratory distress syndrome. Further, the involvement of oxidative damage in atherosclerosis and in ischemia/reperfusion injury in the heart, kidney, CSN or post-operative ischemia/reperfusion injury as well as in thrombosis and embolism makes these disorders liable to intervention by the compounds according to the present invention together with suitable reducing agents. The free radical dependent pathology of ageing and neoplasm development as well as disorders such as Parkinson's and Alzheimer's diseases may also be influenced in a favorable manner by the compounds according to the present invention together with suitable reducing agents. The oxidative damage to tissues caused by radiation, but also by antineoplastic or immunosuppressive agents and other xenobiotics can be prevented or limited by the use of compounds according to the present invention together with suitable reducing agents.

The present invention will now be described in further non-limiting detail and with reference to various Figures and Examples which are described and discussed below.

In the following experiments, organoselenium compound 1 is a compound of the present invention having the following formula:

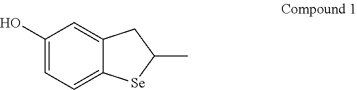

Comparative compounds 2 and 3 are not compounds of the present invention. They have the following formulae:

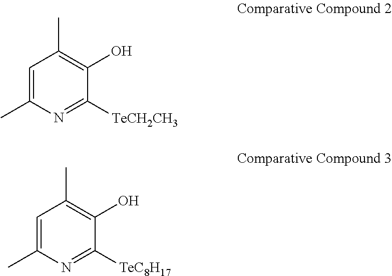

Previously, results according to the two-phase model (discussed above and in J. Org. Chem. 2007, 72, 2583-2595 and Org. Lett. 2008, 10, 21, 4895-4898) indicated that comparative compounds 2 and 3 (which fall outside the definition of the present invention), in comparison to compound 1, are much better chain-breaking antioxidants as determined by the inhibited rate of peroxidation, Rᵢₙₕ, in the presence of N-acetylcysteine in the aqueous phase. The table below shows this data. In addition, both of the comparative compounds have a capacity to act as catalytic preventive antioxidants by decomposing hydroperoxides via redox cycling of tellurium between the oxidation states II (telluride) and IV (telluroxide).

| | Inhibited Rate of Linoleic Acid |
|---|---|
| | Peroxidation (Rᵢₙₕ) for Compounds |
| | Tested in the Two-Phase Model |
| Compound | Rᵢₙₕ (μM h⁻¹) |
| 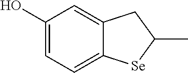 Compound 1 | 69 |
| 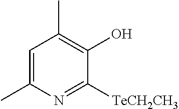 Comparative Compound 2 | 22 |
| 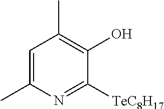 Comparative Compound 3 | 8 |

Based on the results from the two-phase model, it would be difficult even for a person skilled in the art to predict how these compounds would perform as antioxidants in complex biological systems where so many other factors come into play. If anything, the above data rather teaches away from the use of compound 1 as an effective antioxidant.

Surprisingly, as shown in the experiments below, compound 1 is not only highly effective in cellular systems, but is actually significantly more effective than comparative compounds 2 and 3. Thus, the results from the two-phase model cannot always be extrapolated to give a true indication of efficacy in vivo. Furthermore, compound 1 displays excellent results in terms of non-toxicity that could also not be predicted.

It is surprising that the compounds of the present invention work better than comparative compounds, when in the two-phase model they perform less well than the comparative compounds. Furthermore, the inventor has shown that the compounds of the present invention possess the additional advantage of being pharmaceutically acceptable when so many other selenium-containing compounds are toxic.

Furthermore, compounds of the present invention display unexpectedly good results with respect to concentrations needed to induce cell toxicity in human endothelial cells. In experiments described below, cell toxicity was monitored for up to 72 hours in presence of different concentrations of test compounds and it was found that compound 1 was far less toxic than compounds 2 and 3 and also that the concentration needed for toxicity was about two orders of magnitude higher than the concentrations needed for ROS-inhibition. This fact strongly indicates the presence of a therapeutic window well separated from concentrations where the compounds become toxic.

### EXAMPLES

### Example Series 1

### Validation of Effect of Compounds of the Present Invention in Cell Systems with Induced Reactive Oxygen Species (ROS) Production

Previously, the effectiveness and validity in biological systems of compounds of the present invention had not been established. Therefore, the following experiments were carried out to investigate their effect in cell systems with induced ROS (reactive oxygen species) production. These experiments were carried out on the following potential antioxidants: Compound 1 and Comparative Compounds 2 and 3.

The purpose of the experiments was to validate the antioxidative effect of these substances on human neutrophil cell line PLB985, human neutrophils and PBMC (peripheral blood mononuclear cells) and rat neutrophils and PBMC.

Validation was performed by stimulating cells with PMA and fMLF. When treated with the antioxidants, altered level of ROS production from the cells was measured using luminometry. Both intracellular and extracellular effect on ROS on the cells was analyzed using isoluminol (extracellular) and luminol (intracellular when co-administered with SOD and catalase) (Dahlgren et al. “Respiratory burst in human neutrophils”, Journal of Immunological Methods 232 1999; 3-14).

The viability of cells treated with the compounds was also analyzed using a Resazurin based in vitro Toxicology Assay Kit (SigmaAldrich TOX8). Resazurin systems measure the metabolic activity of living cell. Resazurin in its oxidized form (blue) is reduced (red) by living cells and the amount of dye conversion is measured fluorometrically or spectrophotometrically indicating the degree of cytotoxicity.

Thus, the experiments investigate the following in detail:
1) Antioxidative effect against intracellular ROS in viable cells
2) Antioxidative effect against extracellular ROS in viable cells
3) Species differences betweens humans and rats
4) Cell specific effect with respect to monocytes/neutrophils
5) Dependence on NAC for efficacy
6) Cytotoxicity

### MATERIALS AND METHODS

### Reagents

The following reagents were used:
HBSS (Invitrogen-Gibco 14025)
Isoluminol (4-Aminophtalhydrazide) (Sigma A8264). Isoluminol is dissolved in 0.1M NaOH at 10 mg/ml. This stock solution can be kept at RT in darkness for several months.
Luminol (5-amino-2,3-dihydro-1,4-phtalazinedione) (Fluka 09253). Luminol is dissolved in 0.1M NaOH at 10 mg/ml. This stock solution can be kept at RT in darkness for several months.
Catalase from bovine liver (Fluka 60640) 300 000 U/ml
Superoxide Dismutase (SOD) from bovine erythrocytes (Sigma S7571). Dissolved in HBSS to 3000 U/ml and stored in small aliquots at −20° C.
In vitro Toxicology Assay Kit, Resazurin based (Sigma R-6892), stored at 2-8° C.
HRP fraction II (Sigma P8250) diluted in HBSS at 100 units/ml and stored in aliquots at −20° C.
PMA (Sigma P8139), diluted to 1 mg/ml in DMSO and stored in aliquots at −20° C.

PMA is a potent tumor promoter; activates protein kinase C in vivo and in vitro and is a very potent NADPH activator.
fMLF (Sigma F3506), diluted to 4 mg/ml in DMSO and stored in aliquots at −20° C.

fMLF is a potent inducer of leucocyte chemotaxis and macrophage activator. It induces a metabolic burst in macrophages accompanied by an increase in respiratory rate, secretion of lysosomal enzymes, and production of superoxide anion. Receptors that bind formylpeptides are found on phagocytic neutrophils and have recently been identified on cells of the intestinal mucosa.
NAC (N-acetylcysteine). The thiol NAC at 100 micro-M is included as a reducing agent for the test compounds. A 10 mM stock solution of NAC will be prepared in assay buffer, so that that one arm of the experiment can be done with the addition of NAC in the assay.

### Compounds

Compound 1 and Comparative Compounds 2 and 3 were tested. Fresh solutions in DMSO were prepared on the day of experiment.

The compounds were tested in all experiments in dilution series of 120 microM to 0 microM of final concentration.

The NAC concentration was tested in dilution series 200, 100 and 0 microM final concentration.

PMA and fMLF were used in final reaction concentration of 30 ng/ml.

### Plates and Detection

### Luminometry

White 96 well plates (VWR 732-2698 microplate 96 f fluoronunc white) and detection in luminescence detector (FluoStar).

### Spectrophotometry

Transparent 96 well culture plate, detection in Spectra MAX 250 using Soft MAX PRO software.

### Cells

### PBMC and Neutrophils

Human blood was purchased in day-old buffy coats from Blodcentralen, Sahlgrenska Univeristy hospital (Blodcentralen, 031-342 36 54). Rat blood was taken from rats at the central animal facility EBM in Göteborg (Sofia Berntsson 031-7865874, Rosita Olden 031-7865845). Various strains of rats can be used, like Sprague-Dawley, Wistar or Fischer rats. The rats were sacrificed using carbon dioxide and blood was taken by heart puncture. Isolated blood was heparinised to prevent coagulation. Whole blood was mixed with equal volume of room temp. 0.9% NaCl and Dextran 2%, diluted in 0.9% NaCl to a final concentration of 1%. Cells were allowed to sediment for 20-35 min before the upper layer was isolated and transferred to centrifugation tubes, ca 15 ml/tube. Using a syringe with injection needle an equal volume of Ficoll-Paque Plus (GE Healthcare) was added to the bottom of each tube. These were centrifuged at 2000 rpm, 4° C. for 15 min.

PBMCs were isolated from the middle layer using a Pasteur pipette and were transferred to new, pre-chilled tubes. These were diluted in cold HBSS, counted and pelleted. Dilution was carried in proper volume of cold HBSS to get 2 million cells/ml, and storage was on ice.

To isolate the neutrophils, all liquid was removed and the bottom pellet was saved, containing neutrophils and remaining erytrocytes. These cells were washed in 20 ml KRG without Ca²⁺, 900 rpm, 10 min. Erythrocytes were lysed by adding 6 ml dH₂O for 30 seconds. 2 ml 2.4% NaCl in PBS was added, and washing was in KRG without Ca²⁺, ca 10 ml. Centrifuging was at 900 rpm, 4° C., 10 min. The lysation was repeated 2-4 times. Isolated neutrophils were resuspended in proper volume KRG with Ca²⁺ to get 2 million cells/ml.

### PLB985

Human neutrophil cell line PLB985 was grown in RPMI 1640 (Gibco) supplemented with 10% FCS, Penicillin/Streptomycin. The cells were grown at 37° C. at 5% CO₂. The cell are in suspension and when the growth is close to “saturation”. The cells are pelleted and resuspended in new media with the addition of 1.25% DMSO (625 micro- 1/50 ml media). This treatment induces differentiation to neutrophils after 5-8 days (Then, L.; King, A. A. J.; Xiao, Y.; Chanock, S. J.; Orkin, S. H.; Dinauer, M. C. Proc. Natl. Acad. Sci. 1993, 90, 9832-9836) (Tucker, K. A.; Lilly, M. B.; Heck, L.; Rado, T. A. Blood 1987, 70, 372-378). These cells duplicate once every 24 hours. So 1 million cells in 50 mL media will reach saturation after 7 days of culture and be about 1-1.5 million cells/ml. Cells were washed in 20 mL HBSS and resuspended at 2 million cells/ml.

| | Human | | Rat | | |
|---|---|---|---|---|---|
| | Neutro- | Human | Neutro- | Rat | |
| | phils | PBMC | phils | PBMC | PLB985 |
| Dead cells: | 4.9 × 10⁴ | 2.9 × 10⁴ | 1.0 × 10⁴ | 1.4 × 10⁴ | 2.2 × 10⁴ |
| Total cells: | 3.07 × 10⁶ | 6.0 × 10⁶ | 8.25 × 10⁵ | 1.66 × 10⁶ | 1.14 × 10⁶ |
| Viable | 3.02 × 10⁶ | 6.0 × 10⁶ | 8.16 × 10⁵ | 1.65 × 10⁶ | 1.12 × 10⁶ |
| cells: | (98.4%) | (99.9%) | (98.9%) | (99.2%) | (98.2%) |
| Diluted to: | 3 million/ | 4 million/ | 2 million/ | 2 million/ | 4 million/ |
| | ml | ml | ml | ml | ml |

### PROCEDURES

### Working Solution NADPH Activators

PMA and fMLF were used at a working concentration of 120 ng/ml and final reaction concentration of 30 ng/ml. Dilutions to final concentrations were done in HBSS.

### Working Solution Test Antioxidant Compounds

The test compounds were diluted in DMSO to 10 mM concentrations. Further dilutions were made in HBSS. The activity of the test compounds was tested in a dose titration of 120 micro-M to 0 micro-M range of final reaction concentration.

### Working Solution NAC

NAC was diluted in HBSS to 10 mM, final reaction concentrations were 200, 100 and 0 micro-M. Dilution to final reaction concentrations was done in 2× Assay (Isoluminol/Luminol) buffer.

### Isoluminol Assay Buffer

The isoluminol reagent buffer was made of HBSS buffer (with Ca²⁺), Isoluminol and HRP.

| | For 1 | | For 10 | | For 20 | For 40 |
|---|---|---|---|---|---|---|
| Material | plate | | plates | | plates | plates |
| HBSS | 5 | mL | 50 | mL | 100 mL | 200 mL |
| (Invitrogen) | | | | | | |
| Isoluminol | 175 | mikroL | 1750 | mikroL | 3.5 mL | 7 mL |
| (10 mg/ml) | | | | | | |
| HRP II | 175 | mikroL | 1750 | mikroL | 3.5 mL | 7 mL |
| (100 u/ml) | | | | | | |

Isoluminol (10 mg/ml) should be kept dark at RT. HRP should be kept in the fridge, or can be frozen if stored for longer periods. The Isoluminol Assay buffer is quite stable but should be prepared fresh every day or twice daily.

Luminol Assay buffer

The luminol reagent buffer is made of HBSS buffer (with Ca²⁺), luminol and SOD/Catalase.

| | For 1 | | For 10 | | For 20 | | For 40 | |
|---|---|---|---|---|---|---|---|---|
| Material | plate | | plates | | plates | | plates | |
| HBSS | 5 | mL | 50 | mL | 100 | mL | 200 | mL |
| (Invitrogen) | | | | | | | | |
| Luminol | 175 | mikroL | 1750 | mikroL | 3.5 | mL | 7 | mL |
| (10 mg/ml) | | | | | | | | |
| SOD | 100 | mikroL | 1000 | mikroL | 2 | mL | 4 | mL |
| (3000 u/ml) | | | | | | | | |
| Catalase | 400 | mikroL | 4 | mL | 8 | mL | 16 | mL |
| (300 000 u/ml) | | | | | | | | |

Luminol (10 mg/ml) should be kept dark at RT. The Luminol Assay buffer is quite stable but should be prepared fresh every day or twice daily.

The antioxidants and activators were diluted in Isoluminol/Luminol Assay buffers

### Assay Procedure: Luminometry

1) Add 25 microL of antioxidants to assay plate to get proper concentrations in a total reaction volume of 100 micro-L.
2) Add 25 microL of PMA (120 ng/ml) and fMLF (120 ng/ml) to assay plate to get concentration of 30 ng/ml in a total reaction volume of 100 microL.
3) Add 50 microL of cell suspension (2 million cells/ml) to each well. Immediately after addition of cell suspension initiate luminescence measurement; the reaction starts at once when the cells are added to the assay plate. Measurement points are taken at 1-minute intervals for 25 minutes. Data is presented as AUC (Area Under Curve) by a summary of all collected measurements for each well.

### Assay Procedure: Toxicology Analysis

1) 75 micro-l of PLB985 cells (1 million cells/ml) is mixed with 25 micro-l of Compounds 1, 2, 3 and D (480, 120, 15 and 0.48 micro-M) in a 96 well plate in duplicates.
2) 10 micro-L of Resazurin solution is added to each well and the plate is incubated at 37° C. for 3 hours.
3) Spectrophotometrical analysis is performed in plate reader at 600 and 690 nm.

### Results

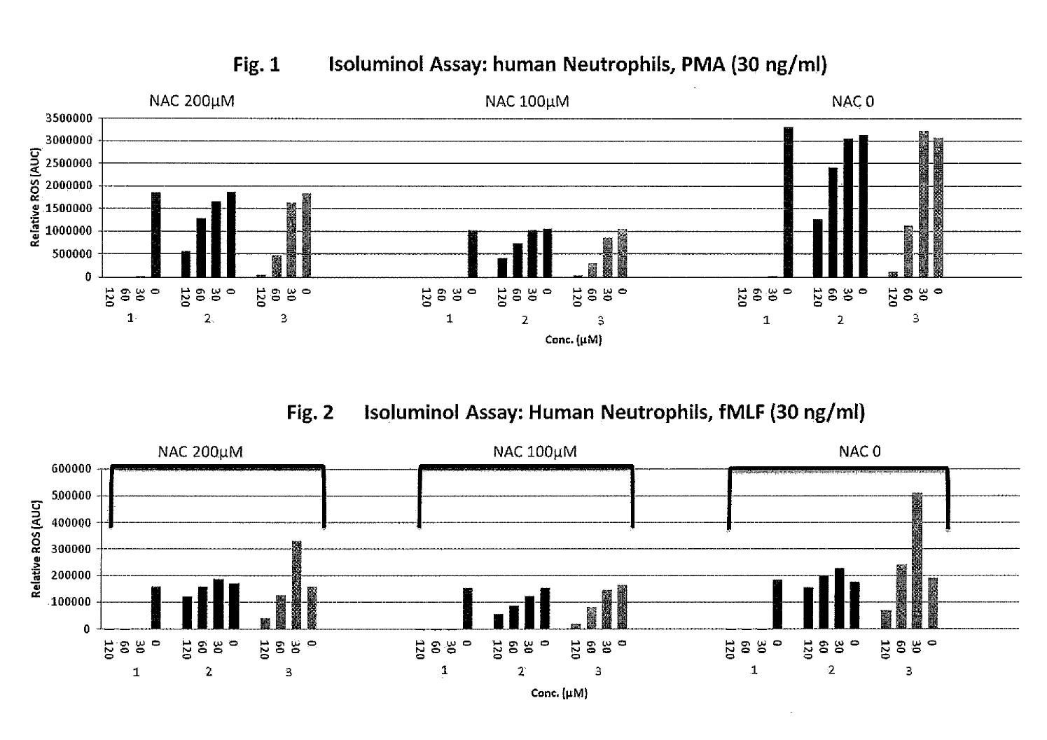

Results are shown in FIGS. 1 to 18 below. All data represent single measurement points. Data is presented as relative AUC (Area Under Curve) by a summary of all collected measurements for each well in plate. Each figure represents the result from a 96 well plate. No normalization for differences between the plates in signal has been made, so direct comparisons between plates are difficult since this is also affected by cell types and time during the day of measurements.

The annotations “1”, “2” and “3” under the bar charts indicate the results in respect of compound 1 and comparative compounds 2 and 3 respectively.

The graphs shown in FIGS. 1 to 18 correspond to the following experiments.

FIG. 1: Extracellular ROS analyzed by Isoluminol induced luminescence in human Neutrophils. ROS induced with PMA (30 ng/ml) with 200, 100 or 0 microM NAC. Concentrations of compounds 120, 60, 30 and 0 microM final concentrations.

FIG. 2: Extracellular ROS analyzed by Isoluminol induced luminescence in human Neutrophils. ROS induced with fMLF (30 ng/ml) with 200, 100 or 0 microM NAC. Concentrations of compounds 120, 60, 30 and 0 microM final concentrations.

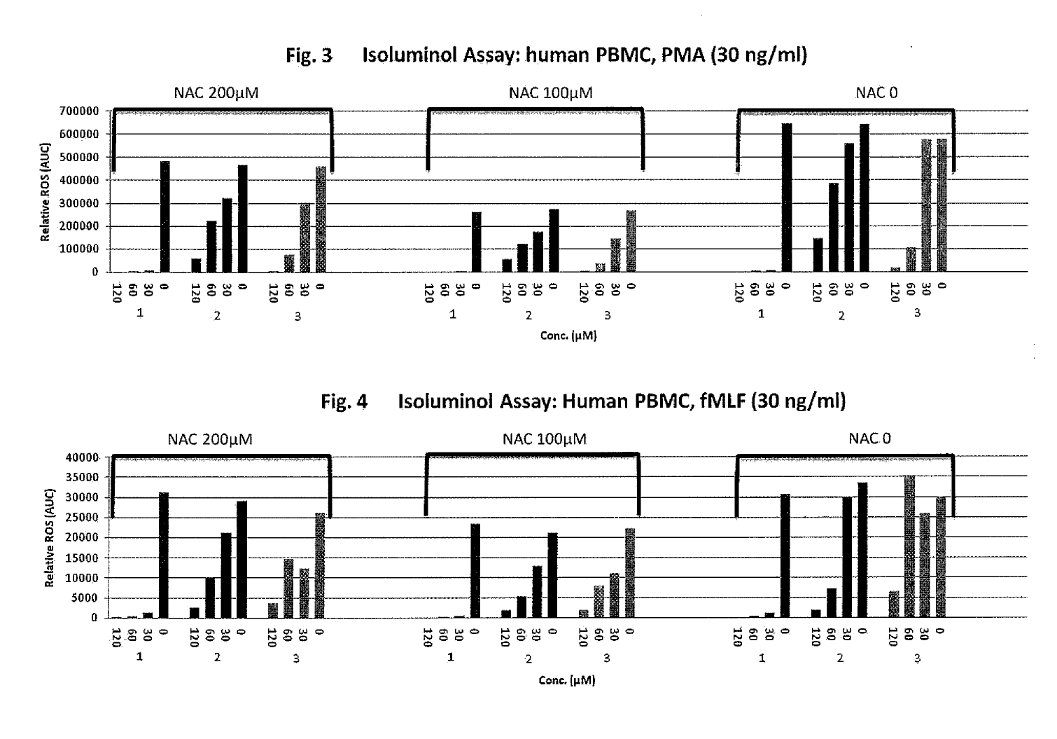

FIG. 3: Extracellular ROS analyzed by Isoluminol induced luminescence in human PBMCs. ROS induced with PMA (30 ng/ml) with 200, 100 or 0 microM NAC. Concentrations of compounds 120, 60, 30 and 0 microM final concentrations.

FIG. 4: Extracellular ROS analyzed by Isoluminol induced luminescence in human PBMCs. ROS induced with fMLF (30 ng/ml) with 200, 100 or 0 microM NAC. Concentrations of compounds 120, 60, 30 and 0 microM final concentrations.

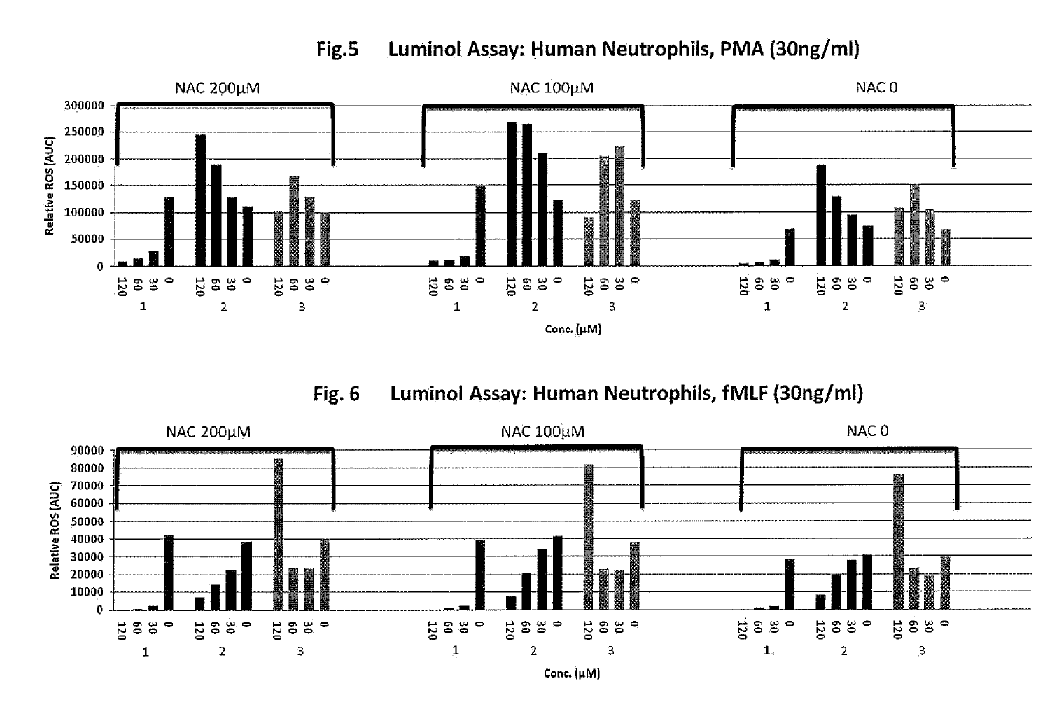

FIG. 5: Intracellular ROS analyzed by Luminol (intracellular ROS) induced luminescence in human Neutrophils. ROS induced with PMA (30 ng/ml) with 200, 100 or 0 microM NAC. Concentrations of compounds 120, 60, 30 and 0 microM final concentrations.

FIG. 6: Intracellular ROS analyzed by Luminol (intracellular ROS) induced luminescence in human Neutrophils. ROS induced with fMLF (30 ng/ml) with 200, 100 or 0 microM NAC. Concentrations of compounds 120, 60, 30 and 0 microM final concentrations.

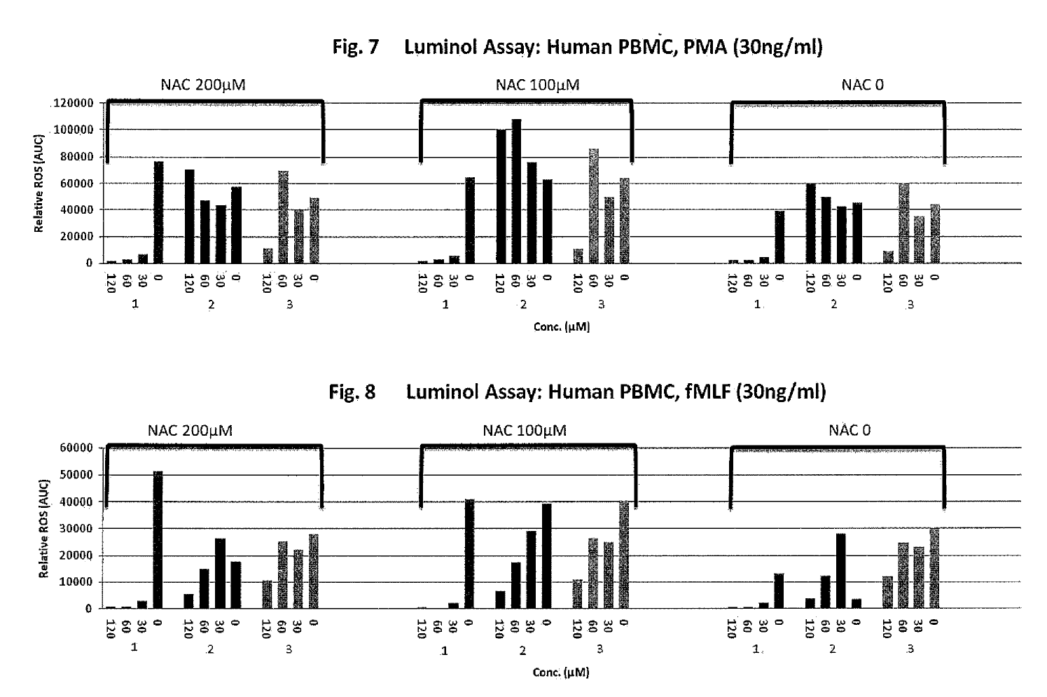

FIG. 7: Intracellular ROS analyzed by Luminol (intracellular ROS) induced luminescence in human PBMCs. ROS induced with PMA (30 ng/ml) with 200, 100 or 0 microM NAC. Concentrations of compounds 120, 60, 30 and 0 microM final concentrations.

FIG. 8: Intracellular ROS analyzed by Luminol (intracellular ROS) induced luminescence in human PBMCs. ROS induced with fMLF (30 ng/ml) with 200, 100 or 0 microM NAC. Concentrations of compounds 120, 60, 30 and 0 microM final concentrations.

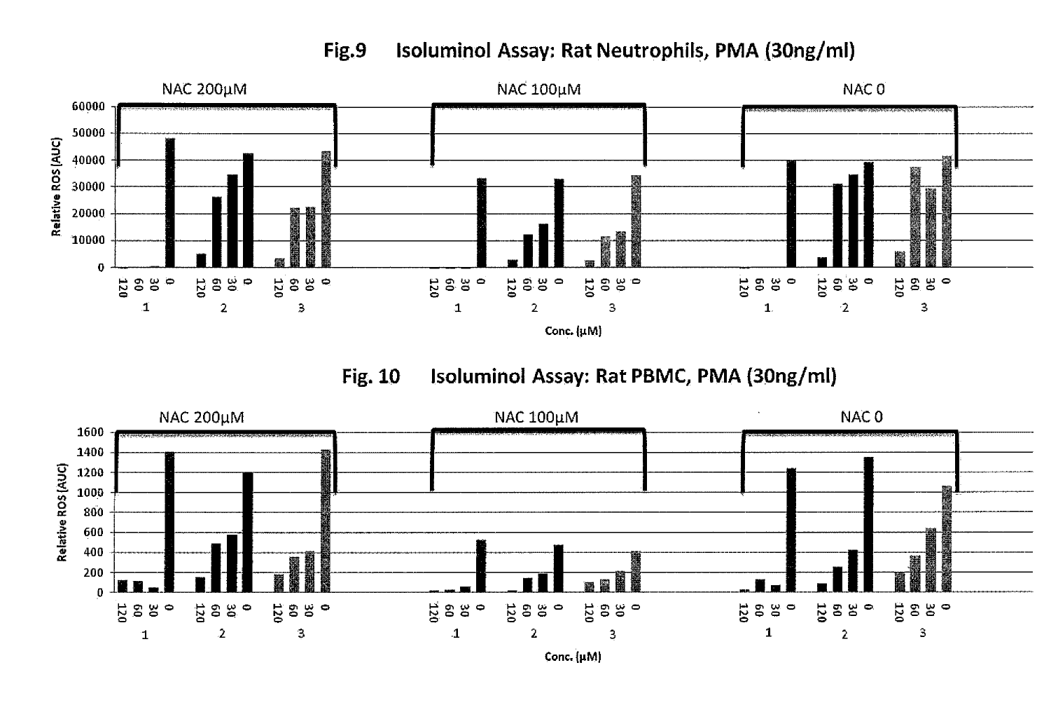

FIG. 9: Extracellular ROS analyzed by Isoluminol induced luminescence in rat Neutrophils. ROS induced with PMA (30 ng/ml) with 200, 100 or 0 microM NAC. Concentrations of compounds 120, 60, 30 and 0 microM final concentrations.

FIG. 10: Extracellular ROS analyzed by Isoluminol induced luminescence in rat PBMCs. ROS induced with PMA (30 ng/ml) with 200, 100 or 0 microM NAC. Concentrations of compounds 120, 60, 30 and 0 microM final concentrations.

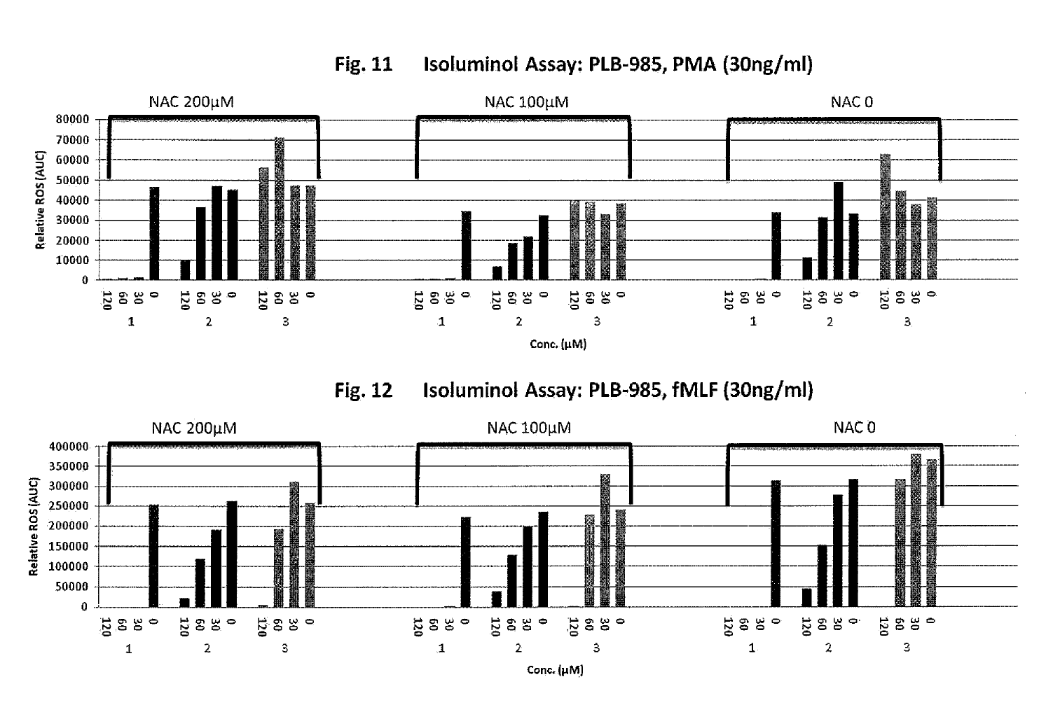

FIG. 11: Extracellular ROS analyzed by Isoluminol induced luminescence in human PLB-985 cell line. ROS induced with PMA (30 ng/ml) with 200, 100 or 0 microM NAC. Concentrations of compounds 120, 60, 30 and 0 microM final concentrations.

FIG. 12: Extracellular ROS analyzed by Isoluminol induced luminescence in human PLB-985 cell line. ROS induced with fMLF (30 ng/ml) with 200, 100 or 0 microM NAC. Concentrations of compounds 120, 60, 30 and 0 microM final concentrations.

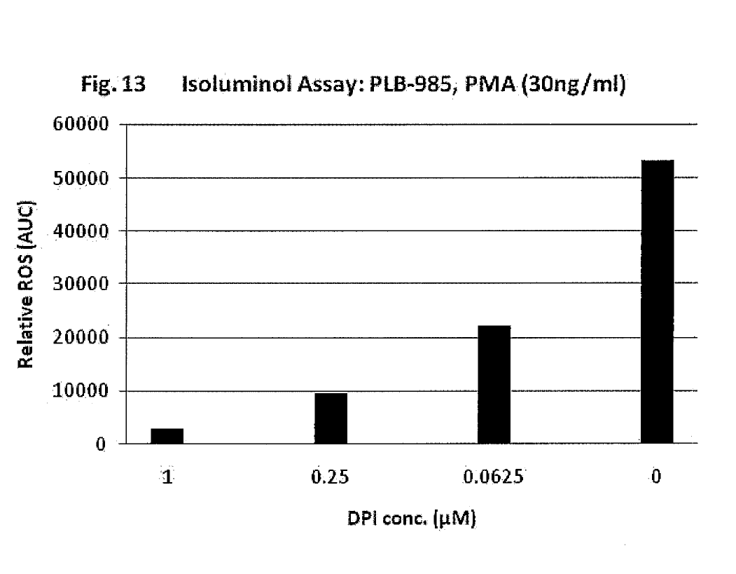

FIG. 13: Extracellular ROS analyzed by Isoluminol induced luminescence in human PLB-985 cell line. ROS induced with PMA (30 ng/ml). Concentrations of DPI 1000, 250, 62 and 0 nM final concentrations. Serve as experimental control to show effect of NADPH oxidase complex blockage with DPI as selective inhibitor.

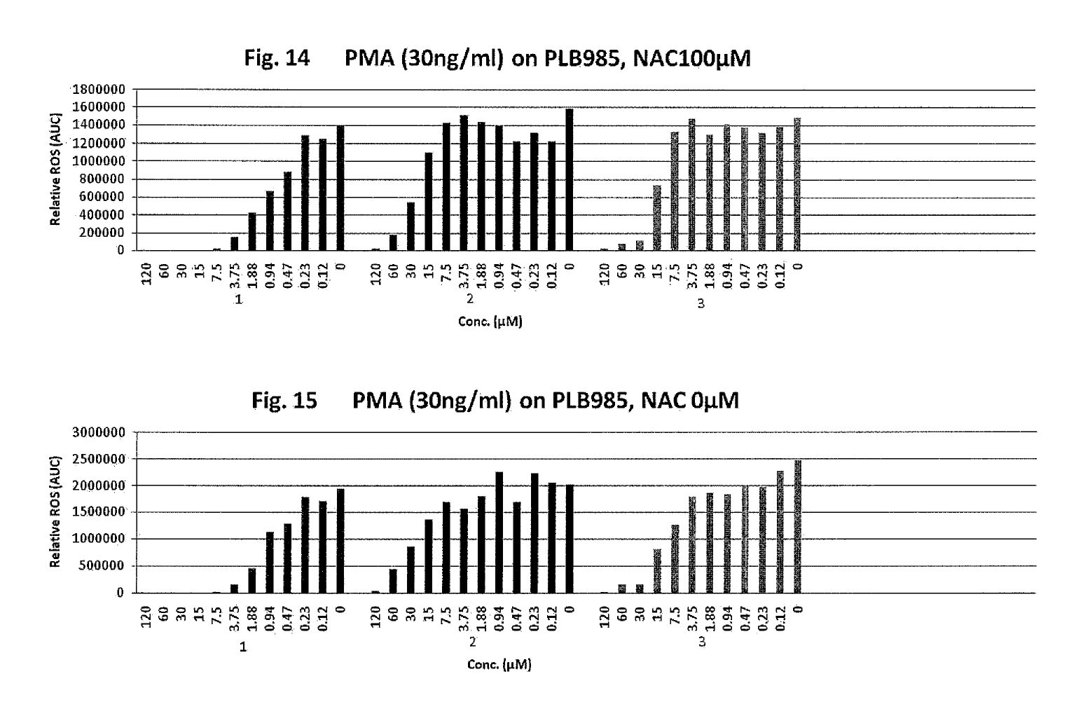

FIG. 14: Extracellular ROS analyzed by Isoluminol induced luminescence in human PLB-985 cell line. ROS induced with PMA (30 ng/ml) with 100 microM NAC. Concentrations of compounds 120; 60; 30; 15; 7.5; 3.75; 1.88; 0.94; 0.47; 0.23; 0.12 and 0 microM final concentrations.

FIG. 15: Extracellular ROS analyzed by Isoluminol induced luminescence in human PLB-985 cell line. ROS induced with PMA (30 ng/ml) with 0 microM NAC. Concentrations of compounds 120; 60; 30; 15; 7.5; 3.75; 1.88; 0.94; 0.47; 0.23; 0.12 and 0 microM final concentrations.

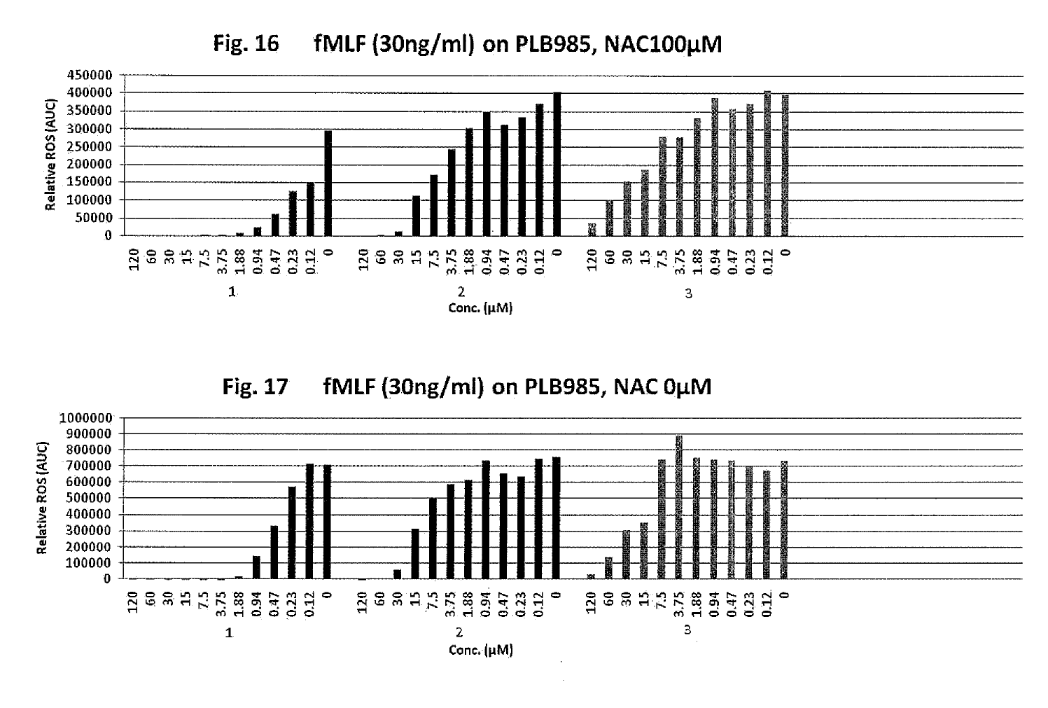

FIG. 16: Extracellular ROS analyzed by Isoluminol induced luminescence in human PLB-985 cell line. ROS induced with fMLF (30 ng/ml) with 100 microM NAC. Concentrations of compounds 120; 60; 30; 15; 7.5; 3.75; 1.88; 0.94; 0.47; 0.23; 0.12 and 0 microM final concentrations.

FIG. 17: Extracellular ROS analyzed by Isoluminol induced luminescence in human PLB-985 cell line. ROS induced with fMLF (30 ng/ml) with 0 microM NAC. Concentrations of compounds 120; 60; 30; 15; 7.5; 3.75; 1.88; 0.94; 0.47; 0.23; 0.12 and 0 microM final concentrations.

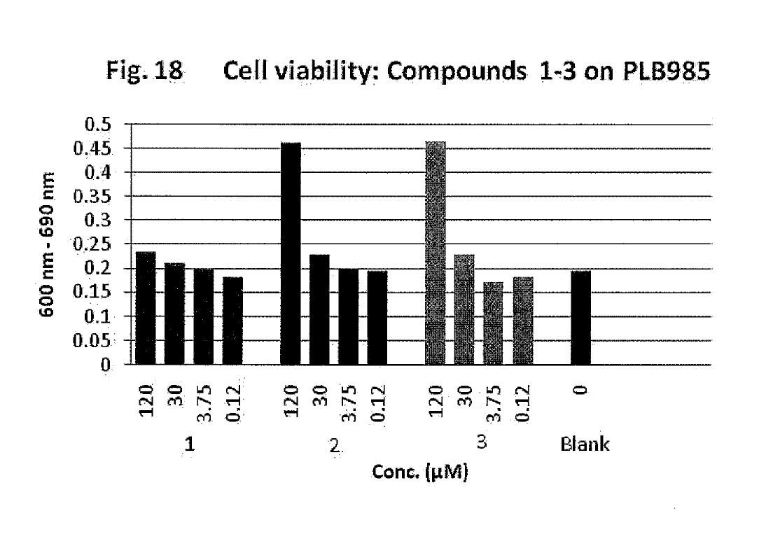

FIG. 18: Cell viability of compounds in human PLB-985 cell line. Concentrations of compounds 120; 60; 30; 3.75; 0.12 and 0 mM final concentrations. Measurement that indicates cell cytotoxicity (In vitro toxicology assay, TOX-8). The bars show increased level of cytotoxicity (analysis of absorbance of 600 nm substracted with absorbance 690 nm).

### Conclusions

Several conclusions can be drawn from the above experiments and the graphs in FIGS. 1 to 18.

### 1) Antioxidative Effect of Intracellular ROS in Viable Cells

Compound 1 has a strong antioxidative effect. Without wishing to be bound by theory, the lesser effect of Compounds 2 and 3 could be due to lower potency or due to problems in passing the cell membrane to the intracellular compartment (FIG. 5-8).

### 2) Antioxidative Effect of Extracellular ROS in Viable Cells

Compound 1 has an antioxidative effect on extra cellular ROS. Compounds 2 and 3 are less potent (FIG. 1-4, 9-12, 14-17).

### 3) Species Difference Human/Rat

We see no species differences between rat and human cell produced ROS (FIG. 1-2 vs 9-10).

### 4) Cell Specific Effect Monocytes/Neutrophiles

We see no cell type specific effect between monocytes and Neutrophils (FIG. 1-2 vs 3-4).

### 5) Dependence of NAC for Efficacy

We see no clear effect of inclusion of NAC (100 or 200 micro-M) or that NAC is critical for the antioxidative effect (differs between all experiments, but clearly no general effect that NAC should be required for the effect of the compounds). It is possible that, as observed in FIGS. 14-17 the general level of ROS in the assay is decreased by inclusion of NAC.

### 6) Cytotoxicity

Compound 1 shows no signs of cytotoxicity. In their highest concentrations (120 μM) Compounds 2 and 3 cause a decrease in cell viability.

In summary, compounds of the present invention are highly and consistently effective in cellular systems.

### Example 2

### Antioxidant Capacity in Aqueous Solution as Determined by an ABTS-Assay

Previously, the antioxidant capacity of compound 1 was tested in lipid (chlorobenzene) phase (J. Org. Chem. 2007, 72, 2583-2595).

In order to investigate its aqueous phase performance, we thought that it would be interesting to determine its Trolox Equivalent Antioxidant Capacity (TEAC) (Free Rad. Biol. Med. 1999, 26, 1231-1237) which has been used to quantify the antioxidant activity of biological fluids, extracts and pure compounds. The TEAC value is defined as the concentration of Trolox which has an equivalent antioxidant potential as a 1 mM solution of the compound under investigation (Free Rad. Biol. Med. 1996, 20, 933-956).

A 2,2′-azinobis(3-ethylbenzothiazoline-6 sulphonic acid radical cation (ABTS•+) assay was used to determine relative TEAC values. To a preformed solution of ABTS•+ in PBS buffer, pH 7.4, with an absorbance slightly above 0.7 were added various amounts (3-30 microM) of organoselenium compound 1, Trolox, sodium ascorbate and N-acetylcysteine. The absorbance at 734 inn was recorded immediately after mixing and then every minute for the next 8 minutes.

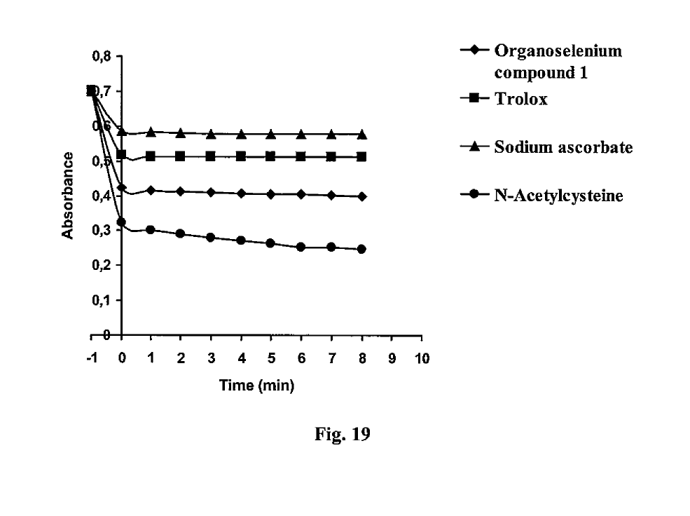

Experiments carried out at 6.08 micro-M concentrations could clearly distinguish the quenching capacity of compound 1 and the three water-soluble reference antioxidants (FIG. 19). Organoselenium compound 1 clearly outperformed both sodium ascorbate and Trolox when it comes to the number of ABTS•+-radicals quenched by each molecule of the antioxidant. The data presented in FIG. 19 would correspond to a TEAC value after 5 min of 1.6 for organoselenium compound 1. Similarly to sodium ascorbate and Trolox, compound 1 quenched ABTS•+-radicals promptly and the oxidation products formed continued to quench radicals only at a very slow rate. With N-acetylcysteine, which showed a higher TEAC value of 2.4, the contribution from the primary oxidation products to further react with ABTS•+-radicals was much larger (Food Chemistry 2003, 80, 409-414).

It is clear from this part of the study that organoselenium compound 1 has a high capacity to quench radicals in an aqueous environment.

FIG. 19 shows absorbance at 734 nm of the ABTS•+-radical cation (47.4 micro-M) with time after addition of organoselenium compound 1, Trolox, sodium ascorbate and N-acetylcysteine (each 6.08 micro-M) in aqueous PBS-buffer.

### Experimental Method

Antioxidant activity as determined by the capacity of the antioxidant to decolorize the ABTS⁺ radical cation was determined essentially as described in the literature (Acc. Chem. Res. 1986, 19, 194-201). ABTS, (2,2′-azinobis(3-ethylbenzothiazoline-6-sulfonic acid diammonium salt; 5.76 mg, 0.0105 mmol), was allowed to react with less than the stoichiometric amount of potassium persulfate (1.00 mg, 0.0037 mmol) in water (1.5 mL) to produce, after 15 h, a stock solution of the corresponding radical cation with a stable absorbance. This stock solution (0.0200 mL), diluted with PBS buffer-pH 7.4 (1.48 mL), showed an absorbance of 0.711 at 734 nm. Using an extinction coefficient of 1.5×104 for the radical cation ABTS⁺ in water, this value corresponds to a 47.4 microM concentration of the ABTS⁺ radical cation. In a typical experiment, the antioxidant (1.52 micro-L of a 3.00 mM solution in MeOH or water; final concentration=6.08 micro-M) was syringed into 0.750 mL of the ABTS⁺ radical cation solution. After initial mixing, the absorbance was recorded every minute for the next eight minutes. FIG. 19 shows a plot of absorbance versus time for experiments carried out with Trolox, organoselenium compound 1 and sodium ascorbate.

### Example 3

### Assessment of Cell Viability with Alamar Blue and LDH Assays

Alamar Blue is a non-toxic metabolic indicator for viable cells. Following uptake, the dye becomes reduced and causes a change in the colour of the cell. The colour change correlates reasonably well with the number of living cells in the sample.

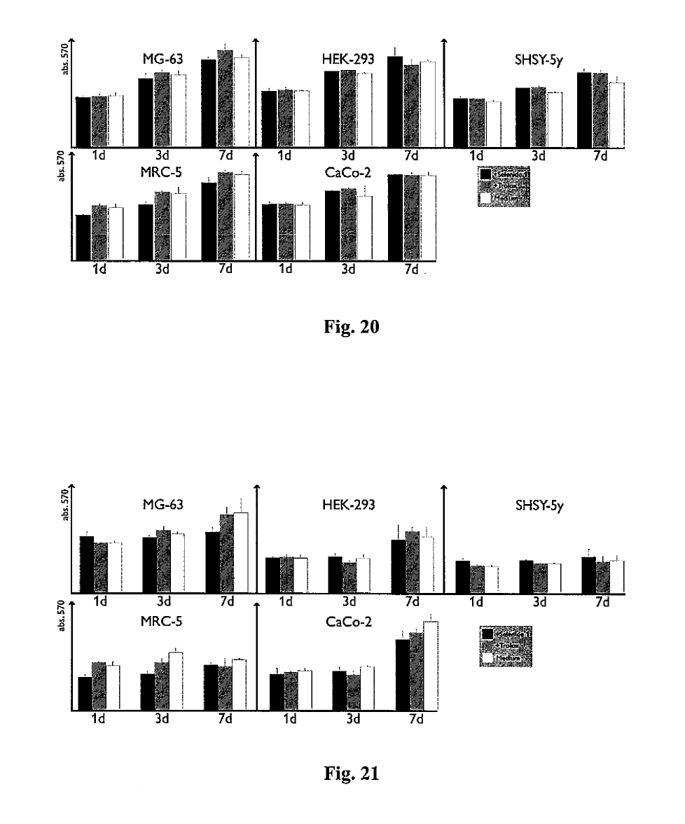

As can be seen in FIG. 20, the Alamar Blue assay showed normal cell growth patterns with cell numbers increasing from day 1 to day 7 both in the presence of selenide 1 (60 micro-M) and Trolox (60 micro-M) which was used as an antioxidant reference. This pattern was observed more or less independently of cell type or antioxidant additive. The broad selection of human cell types in addition to the relatively high concentration of antioxidants clearly indicate that normal cellular proliferation is not affected. Considerably higher concentrations of selenide 1 were also tested on these cell-lines. These experiments indicated no sign of cell death or decreased proliferation until concentrations as high as 235 micro-M (data not shown).

FIG. 20 shows relative viability of MG-63, HEK-293, SHSY-5y, MRC-5 and CaCo-2-cells in the presence/absence of 60 micro-M of selenide 1 or Trolox as determined by alamar blue measurements (absorbance at 570 nm) after 1 day (1 d), 3 days (3 d), and 7 days (7 d).

Cell death and plasma membrane damage was also assayed by measuring the release of lactate dehydrogenase (LDH), a stable cytoplasmic enzyme present in most cells. In the kit provided by Sigma, LDH participates in a reaction which converts a yellow tetrazolium salt into a red formazan-class dye. The amount of formazan is directly proportional to the amount of LDH in the culture, which is in turn proportional to the number of dead or damaged cells.

The LDH assay correlated well with the proliferation data, that is, no significant signs of toxicity for any of the cell types could be seen as a consequence of addition of selenide 1 or Trolox at 60 micro-M (FIG. 21).

FIG. 21 shows relative cytotoxicity caused by 60 micro-M of selenide 1 or Trolox in MG-63, HEK-293, SHSY-5y, MRC-5 and CaCo-2-cells as determined by LDH measurements (absorbance of formazan at 570 nm) after 1 day (1 d), 3 days (3 d), and 7 days (7 d).

### Cell Cultures

MG-63, SHSY-5Y, MRC-5, Caco-2 and HEK 293 were cultured in DMEM:F12 (Sigma-Aldrich) supplemented with 10% FCS, 100 IU penicillin/ml, 100 μg streptomycin/ml, 2 nM L-glutamine and 1% non essential amino acids at 37° C., 5% CO2 in a humidified atmosphere.

The THP-1 cells were grown in RPMI-1640 (GIBCO®) supplemented with 10% FCS, 100 IU penicillin/ml, 100 μg streptomycin/ml, 2 nM L-glutamin and 1% non essential amino acids at 37° C., 5% CO2 in a humidified atmosphere.

### Cell Proliferation and Cytotoxicity Assays

MG-63, SHSY-5Y, MRC-5, Caco-2 and HEK 293 were harvested using trypsin-EDTA treatment. The cells were centrifuged at 400 g for 5 min after which they were resuspended in phenol red free Alpha medium (GIBCO®) containing 10% FCS, 100 IU penicillin/ml, 100 μg streptomycin/ml, 2 nM L-glutamin and 1% non essential amino acids. 30 000 cells/well (of each cell type) and 60 micro-M of the antioxidants Trolox or SeOH were added to a 24 well plate in triplicate samples. Cells w/o antioxidant were used as controls.

### Alamar Blue

Culture medium was removed from the wells after 1, 3 and 7 days and replaced by 0.5 ml Alamar Blue stock solution (Serotec) diluted 1:10 in Hanks balanced salt solution (HBSS) and incubated at 37° C., 5% CO₂ in a humidified atmosphere for 2.5 h. Alamar Blue is a non-toxic metabolic indicator for viable cells. Upon uptake into the cell the dye becomes reduced and changes colour. The colour change correlates approximately with the number of living cells in the sample. Aliquotes of 100 micro-1 from each well were transferred to a 96-well plate and the absorbance was read at 570 nm using a multiscan MS spectrophotometer (Labsystems).

### LDH

Culture medium was removed from the wells after 1, 3 and 7 days and used for cytotoxicity measurements using an LDH in vitro toxicology assay kit (Sigma®) according to the manufacturer's protocol. Cell death and plasma membrane damage can be assayed measuring the release of lactate dehydrogenase (LDH), a stable cytoplasmic enzyme present in most cells. LDH participates in a coupled reaction, which converts a yellow tetrazolium salt into a red, formazan-class dye. The amount of formazan is directly proportional to the amount of LDH in the culture, which is in turn proportional to the number of dead or damaged cells. Absorbance was read at 570 nm using a multiscan MS spectrophotometer (Labsystems).

### Example 4

### Effects of Selenide 1 ON Free Radical Scavenging as Determined by ROS Production in Neutrophils and Macrophages

We investigated the capacity of the material to quench ROS produced by freshly isolated human neutrophils or THP-1 cells (human acute monocytic leukemia cell line) stimulated with PMA (Phorbol Myristate Acetate). The antioxidant capacity of selenide 1 was again compared to those of Trolox, a water soluble derivative of vitamin E, which is commonly used in biological and biochemical applications to reduce oxidative stress or damage.

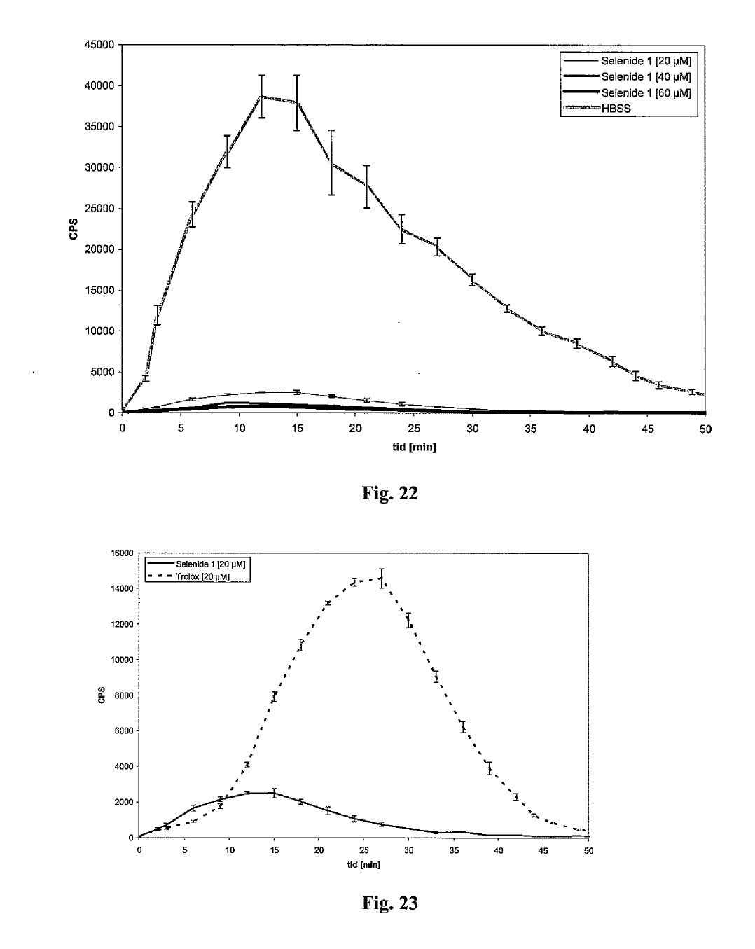

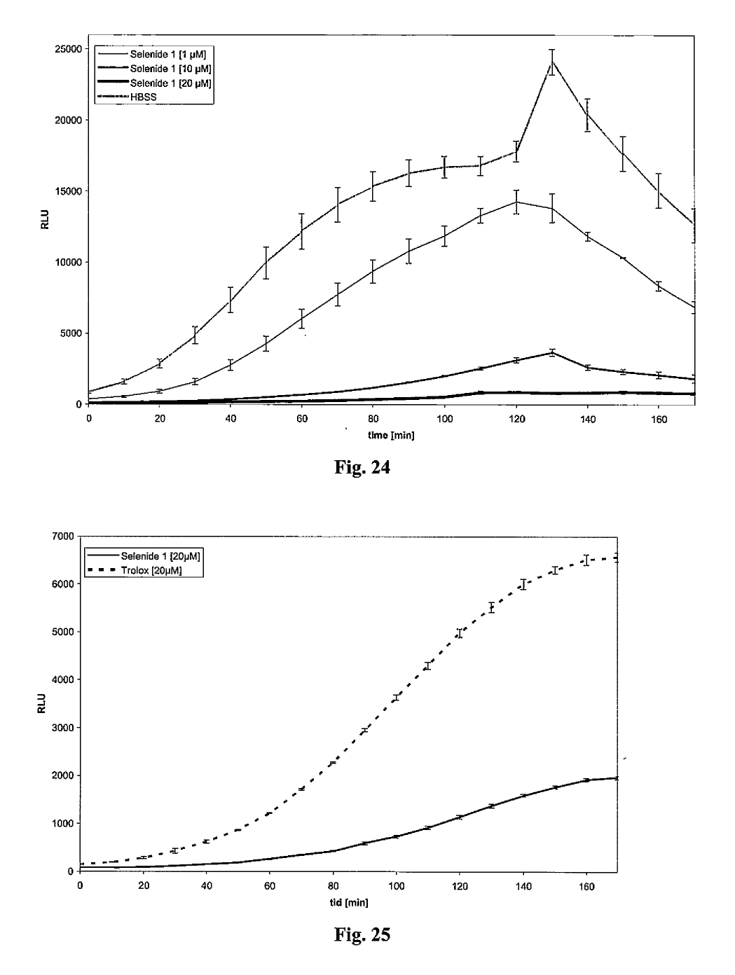

In a first attempt total ROS (i.e., both extra- and intracellular) production was measured using modified luminol enhanced chemiluminescence (CL). As can be seen in FIG. 22 (neutrophils) and FIG. 24 (macrophages), selenide 1 showed a clear, dose dependent, quenching/inhibiting effect on ROS production, independent of cell source. FIGS. 22 and 24 demonstrates that selenide 1 is considerably more effective than Trolox in quenching ROS produced from both neutrophils and THP-1. By using both neutrophils and monocyte/macrophages we were able to test the antioxidant capacity for both short and long term exposure to ROS.

FIG. 22 shows chemoluminescence measured in counts per second (CPS) for human neutrophiles exposed to various amounts of selenide 1 (20, 40, 60 micro-M) in comparison with a medium control (PMM+HESS). Error-bars are indicated. The upper curve corresponds to the HESS results. The lower curves, which are close to each other, correspond to the selenide 1 results at the three concentrations.

FIG. 23 shows chemoluminescence measured in counts per second (CPS) for human neutrophiles exposed to selenide 1 (20 micro-M) or Trolox (20 micro-M). Error-bars are indicated.

FIG. 24 shows chemiluminescence profiles for THP-1 cells exposed to different concentrations of selenide 1 (1, 10 and 20 micro-M) measured in relative chemiluminescence units (RLU). From top to bottom, the curves correspond to

HBSS, selenide 1 at 1 micro-M, selenide 1 at 10 micro-M, and selenide 1 at 20 micro-M, respectively.

FIG. 25 shows chemiluminescence profiles for THP-1 cells exposed to selenide 1 (20 micro-M) and Trolox (20 micro-M) measured in relative chemiluminescence units (RLU).

### Chemiluminescence (CL) Experimental Details

### Neutrophil Isolation

Neutrophils were isolated from heparinized blood of apparently healthy blood donors (Academic Hospital, Uppsala, Sweden) following a routine dextran sedimentation method essentially as described by H\dot{a}lansson and Venge (Scandinavian Journal of Immunology 1980, 11, 271-282). To each of three test tubes containing 5 ml heparinized blood, dextran (T500, Pharmacia, Uppsala, Sweden) was added (final concentration 1%) and the sedimentation was allowed to proceed for 30 min at room temperature. The neutrophil rich supernatant was harvested, pooled and centrifuged at 160 g for 5 min. The pellet was washed twice with 0.9% NaCl. Contaminating erythrocytes were lysed by a 30 s exposure to water (Milli Q) after which 3.6% NaCl was added to reach a final concentration of 0.9%. The obtained suspension was then centrifuged for 5 min at 160 g. Finally, the pellet was resuspended in Gey's buffer and the average content of neutrophils (approximately 90%) was calculated using Türks staining and hemocytometer method. The experiments were performed within 1-2 h after neutrophil isolation.

### Neutrophils

The generation of oxygen free radicals over time from neutrophils was monitored for 40 minutes in 24 well white optiplates (Greiner) using a Wallac Victor (Zweifel, H; “Stabilization of Polymeric Materials”, Springer Berlin, Germany, 1997) in the luminescence mode.

Approximately 300 000 neutrophils diluted in HBSS containing different concentrations of the antioxidants 1 and Trolox were added per well to a 24 well plate together with 100 micro-M luminol (5-amino-2,3-dihydro-1,4 phtallazinedione), 0.17 M NaOH and 6.4 U/ml horseradish peroxidase (HRP) (Research Trends 1991, 427-443). Luminescence intensity was read every 5 min. The measurements were commonly performed on quadruplicate samples.

### THP-1

The generation of oxygen free radicals over time from macrophages (THP-1) was monitored for 180 minutes in 96-well white optiplates (Greiner) using a using a TECAN reader and Diogenes chemiluminescence kit (National Diagnostics). Approximately 200 000 THP-1 cells diluted in HBSS containing different concentrations of the antioxidants 1 and Trolox were added per well to a 96 well plate together with the Diogenes reagent 20 v/% (following the manufacturers recommendations) and 40 micro-M Phorbol Myristate Acetate (PMA). Luminescence intensity was read every 6 min. The measurements were commonly performed on quadruplicate samples

### Example 5

### In Vitro Toxicology Test in Human Umbilical Vein Endothelial Cells

Further experiments were carried out in order to investigate the toxic potential of Compound 1 and Comparative Compounds 2 and 3 on primary endothelial cells derived from human umbilical veins.

The experiments on human umbilical vein endothelial cells described below can be summarised as follows. Compound 1 was toxic at 500 μM after 24 hrs of incubation and at 200 μM after 72 hrs. Comparative Compound 2 was toxic to the cells at 200 after 48 hrs and at 500 μM after 24 hrs. Comparative Compound 3 demonstrated toxicity after only 24 hrs of incubation at 100 μM.

### Equipment

Laminar air flow bench
37° C./6% CO₂ cell incubator
ELISA reader

### Materials

Human umbilical vein endothelial cells pooled, HUVECp (Cascade Biologics, Lot#200706-891)
Medium 200 with supplement (Cascade Biologics)
Compound 1 (MW: 213 g/mol) dissolved in DMSO, concentration 0.1M
Comparative Compound 2 (MW: 278.6 g/mol) dissolved in DMSO, concentration 0.1M
Comparative Compound 3 (MW: 362.6 g/mol) dissolved in DMSO, concentration 0.1M
XTT based (2,3-bis[2-Methoxy-4-nitro-5-sulphophenyl]-2H-tetrazolium-5-carboxyanilide inner salt) toxicology assay (Sigma Aldrich, lot#078K8402) [Roehm, N W., Rodgers, G H., Hatfield, S M., Glasebrook, A L. An improved colorimetric assay for cell proliferation and viability utilizing the tetrazolium salt XTT. J Immunol Methods, 1991. Sep. 13; 142(2):257-65]

### Experimental Procedure

### Seeding of Cells

HUVECp were seeded according to the manufacturer's instructions and subcultured when reaching at least 80% confluence. Medium 200 supplemented with fetal bovine serum, fibroblast growth factor, hydrocortisone, epidermal growth factor, penicillin, streptavidine and gentamicin was utilized.

### Assay Set-Up

Cells were harvested and resuspended in supplemented medium 200 without phenol red. 50 000 cells were added in duplicates to 96-well plates. The compounds were tested on HUVECs according to the following concentrations.

### Final Concentration of Compound 1 and Comparative Compounds 2 and 3 in Test Plates

| | | |
|---|---|---|
| 1 | 1 | μM |
| 2 | 10 | μM |
| 3 | 50 | μM |
| 4 | 100 | μM |
| 5 | 200 | μM |
| 6 | 500 | μM |

The compounds were diluted in Medium 200 from the 0.1M stock solution. The toxic effects of the compounds were tested after 24 hrs, 48 hrs and 72 hrs.

As a negative control, cells incubated with medium containing 0.5% DMSO (corresponding to the highest DMSO concentration above) was used. As a positive control, cells were incubated in the presence of 20% EtOH.

After the incubation with the compounds, the wells were washed with PBS to remove substances which could interfere with the assay. 40 μl of fresh medium 200 without phenol red was added together with 10 μl XTT substance (according to the manufacturer's instructions). The plates were incubated for 8 hrs and the colour development was monitored by measuring the absorbance at 450 nm.

### Results

By visually looking at the cells under the microscope and estimating the dislodging from the surface, the state of the cells was evaluated. Cells incubated with the highest concentration of Compound 1 (500 μM) were affected after 24 hrs. Comparative Compound 2 affected cells at 200 μM after 48 hrs, but only at 500 μM after 24 hrs. For Comparative Compound 3, cells were affected already at 100 μM after 24 hrs.

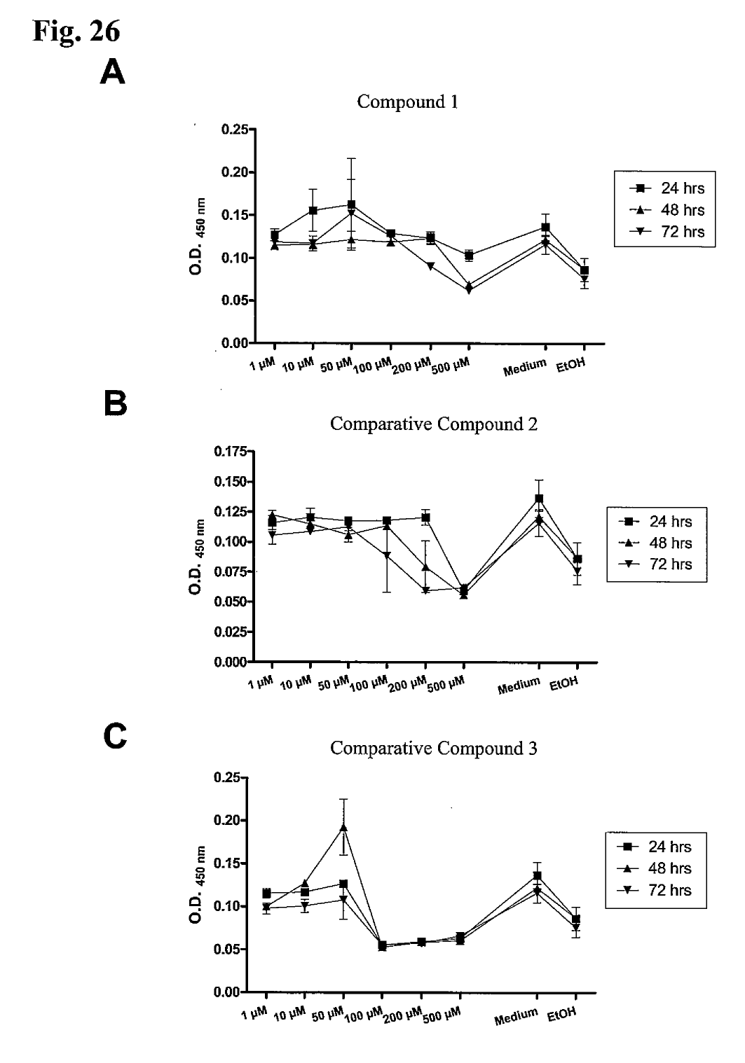

FIG. 26 shows absorbance at 450 nm after incubation of HUVECs with Compound 1 and Comparative Compounds 2 and 3 at various concentrations and for various times. After incubation, the number of viable cells was assessed by observing the amount of metabolized XTT (2,3-bis[2-methoxy-4-nitro-5-sulphophenyl]-2H-tetrazolium-5-carboxyanilide inner salt).

When measuring the absorbance at 450 nm after incubation with the XTT compound, Compound 1 demonstrated toxic properties at 200 μM after 72 hrs, but only at 500 μM after 24-48 hrs (FIG. 26A). A concentration of 200 μM Comparative Compound 2 induced cell death after 48 hrs. After 24 hrs, only the highest concentration of Comparative Compound 2, 500 μM, was toxic (FIG. 26B). Comparative Compound 3 was the most toxic compound. An effect on cell viability was seen already after 24 hrs at 100 μM concentration (FIG. 26C). The higher absorbance for 50 μM Comparative Compound 3 after 48 hrs is most likely an artefact of the assay since only one of the duplicates had a higher absorbance, but there was no visual difference between them. The toxic properties of the three Catanox compounds are summarized in the following table.

| | 24 hrs | 48 hrs | 72 hrs |
|---|---|---|---|
| Compound 1 | 500 μM | 500 μM | 200 μM |
| Comparative Compound 2 | 500 μM | 200 μM | 200 μM |
| Comparative Compound 3 | 100 μM | 100 μM | 100 μM |

### Conclusions

The results of the viability measurements coincide with the visual observations made. Comparative Compound 3 was the most toxic compound demonstrating toxic properties at 100 μM concentrations already after 24 hrs of incubation. Comparative Compound 2 seems to be slightly more toxic than Compounds 1; cells were affected at 200 μM after 48 hrs of incubation. Compound 1 was toxic at 200 μM only after 72 hrs of incubation.

### Further Experiments and Results

Some further compounds were tested as follows:

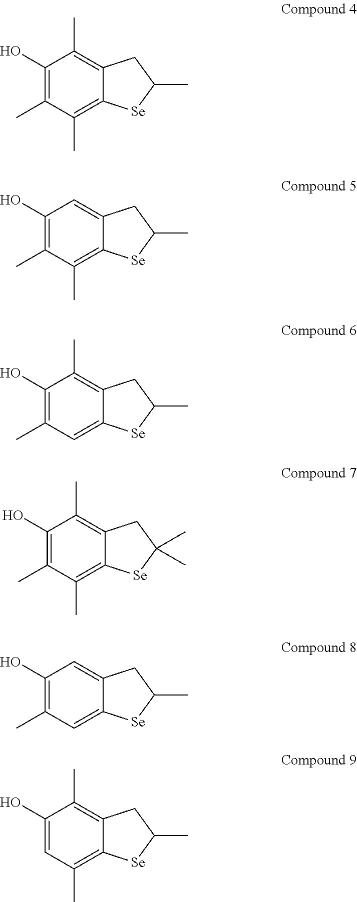

### Example Series 6

### Further Validation of Effect of Compounds of the Present Invention in Cell Systems with Induced Reactive Oxygen Species (ROS) Production

Compounds 4 to 9 were tested in a similar manner to that described under “example series 1”. In series 6, DTT was used instead of NAC. Results are also shown below for compound 1 using DTT in place of NAC.

The antioxidative effect of these substances on human neutrophil cell line PLB985 stimulated with PMA with and without addition of DDT was validated. When treated with the antioxidants, altered level of extracellular ROS production from the cells was measured using luminometry. (Dahlgren et al., Journal of Immunological methods, 1999, cited previously)

This analysis determined the effect of the test compounds:

1) Antioxidative effect of extracellular ROS in viable cells
2) Dependence of DTT for efficacy
Reagents used were as follows:
HBSS
Isoluminol (4-Aminophtalhydrazide) (Sigma A8264). Isoluminol is dissolved in 0.1M NaOH at 10 mg/ml. This stock solution can be kept at RT in darkness for several months.
HRP fraction II (Sigma P8250) diluted in HBSS at 100 units/ml and stored in aliquots at −20° C.
PMA (Sigma P8139), diluted to 1 mg/ml in DMSO and stored in aliquots at −20° C. PMA is a potent tumor promoter; activates protein kinase C in vivo and in vitro and is a very potent NADPH activator.
Dithiothreitol (DTT) at 100 mM was included as a catalyst for the antioxidant capacity of the test compounds. A 10 mM stock solution of DTT was prepared in DMSO, so that that one arm of the experiment could be done with the addition of DTT in the assay.

The compounds were coded with unique numbers and the identity of the structures of the compounds was not disclosed to the researchers during the experiment. The compounds were supplied as dry powder of 1-2 mg each. Two (2) vials of each test compound were supplied so that the test compound could be diluted fresh in DMSO for each days experiments.

| Compound | Molecular weight |
|---|---|
| 1 | 213.1 g/mol |
| 4 | 255.2 g/mol |
| 5 | 241.2 g/mol |
| 6 | 241.2 g/mol |
| 7 | 269.3 g/mol |
| 8 | 227.2 g/mol |
| 9 | 241.2 g/mol |
| Trolox | 250.3 g/mol |
| DTT | 154.25 g/mol |

The compounds were tested in dilution series of 120 μM to 0 μM as final concentration. DTT were tested at 100 μM as final concentration. Of PMA a final reaction concentration of 30 ng/ml were used.

All samples were analyzed in duplicates and presented as mean values of ROS production. Control samples (0) were wells analysed without addition of compound, while control (blank) wells were analysed without NADPH oxidase stimulation (PMA) or Catallox compound added.

Plates and Detection: White 96 well plates (VWR 732-2698 microplate 96 f fluoronunc white) and detection in luminescence detector (FluoStar).

### Cells:

### PLB985

Human neutrophil cell line PLB985 were grown in RPMI 1640 (Lonza) supplemented with 10% FCS, Penicillin/Streptomycin. The cells were grown at 37° C. at 5% CO2. The cell were in suspension and when the growth was close to “saturation” the cells were pelleted and resuspended in new media with the addition of 1.25% DMSO (625 ul/50 ml media). This treatment induced differentiation to neutrophils after 5-8 days (Zhen PNAS, 1993) (Tucker Blood 1987). These cells duplicate once every 24 hours. So 1 million cells in 50 mL media will reach saturation after 7 days of culture and be about 1-1.5 million cells/ml. Cells are washed in 20 mL HBSS and resuspended at 2 million/ml.

### PLB985 091116

Dead Cells No dead cells counted

### Total Cells: 54×10⁶

### Viable Cells: 54×10⁶ (99%)

Diluted to: 4 million/ml in 13.5 ml HBSS

### Procedures:

### Working Solution NADPH Activators

PMA was used at a working concentration of 120 ng/ml and final reaction concentration of 30 ng/ml. Dilutions to final concentrations were done in 2× Isoluminol Assay Buffer.

### Working Solution Test Antioxidant Compounds

The test compounds were diluted in DMSO to 10 mM concentrations. Further dilutions were made in HBSS. The activity of the test compounds were tested in a dose titration of 120 μM to 0 μM range of final reaction concentration.

### Working Solution DTT

DTT were diluted in DMSO to 10 mM, final reaction concentration were 100 μM. Dilution to final reaction concentrations were done in HBSS.

### Isoluminol Assay Buffer

The isoluminol Assay buffer was made of HBSS buffer (with Ca2+), Isoluminol and HRP.

| | For 1 | | For 10 | | For 20 | For 40 |
|---|---|---|---|---|---|---|
| Material | plate | | plates | | plates | plates |
| HBSS | 5 | mL | 50 | mL | 100 mL | 200 mL |
| Isoluminol | 175 | microL | 1750 | microL | 3.5 mL | 7 mL |
| (10 mg/ml) | | | | | | |
| HRP II | 175 | microL | 1750 | microL | 3.5 mL | 7 mL |
| (100 u/ml) | | | | | | |

Isoluminol (10 mg/ml) should be kept dark at RT. HRP should be kept in the fridge, or can be frozen if stored for longer periods. The Isoluminol Assay buffer is quite stable but should be prepared fresh every day or twice daily.

### Assay Procedure

1) 25 μL of antioxidants were added to assay plate to get proper concentrations (120-0 μM) in a total reaction volume of 1004.
2) 25 μL of PMA (120 ng/ml) were added to assay plate to get concentration of 30 ng/ml in a total reaction volume of 100 μL.
3) 25 μL of DTT (400 μM) were added to assay plate to get concentration of 100 μM in a total reaction volume of 100 μL.
4) 25 μL of cell suspension (4 milj. cells/ml) were added to each well.

Immediately after addition of cell suspension initiate Luminescence measurement, the reaction starts at once when the cells are added to the assay plate.

Measurement points are taken at 1-minute intervals for 25 minutes.

Trolox was also tested in this system.

### Results

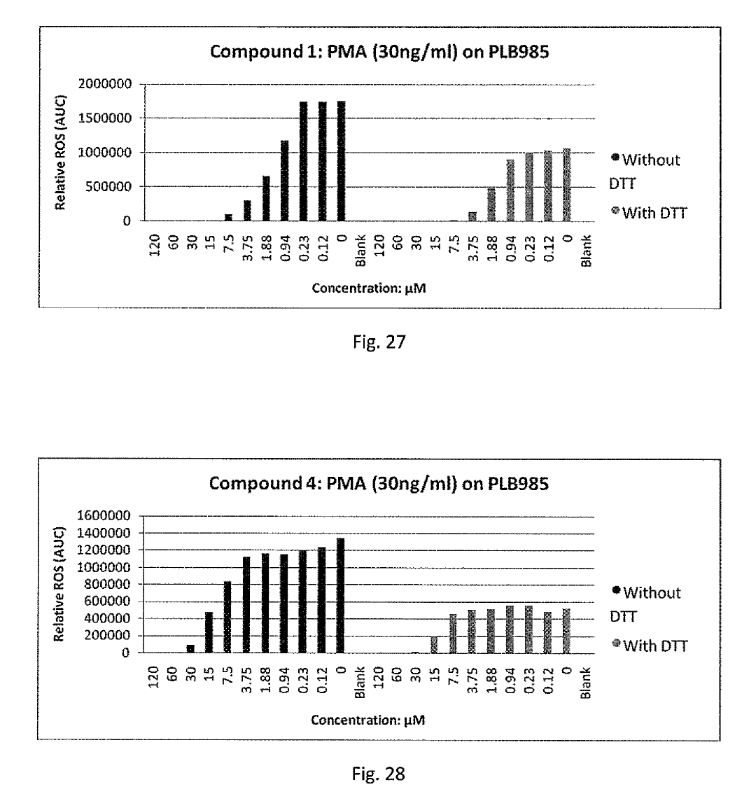

Results are shown in FIGS. 27 to 34 below. Results are shown as Area Under Curve (AUC) values, which are an addition of the values from each measurements point and represent an absolute production of ROS detected in the presence of Catanox agent. Analyses were done with the addition of reducing agent DTT (left bars) and without the addition of DTT (right bars).

The graphs shown in FIGS. 27 to 34 correspond to the following experiments:

FIG. 27: Extracellular ROS analyzed by Isoluminol induced luminescence in human PLB-985 cell line. ROS induced with PMA (30 ng/L). Concentrations of compound 1: 120; 60; 30; 15; 7.5; 3.75; 1.88; 0.94; 0.23; 0.12; and 0 microM. Left panel: Without DTT. Right panel: With DTT 100 microM.

FIG. 28: Extracellular ROS analyzed by Isoluminol induced luminescence in human PLB-985 cell line. ROS induced with PMA (30 ng/L). Concentrations of compound 4: 120; 60; 30; 15; 7.5; 3.75; 1.88; 0.94; 0.23; 0.12; and 0 microM. Left panel: Without DTT. Right panel: With DTT 100 microM.

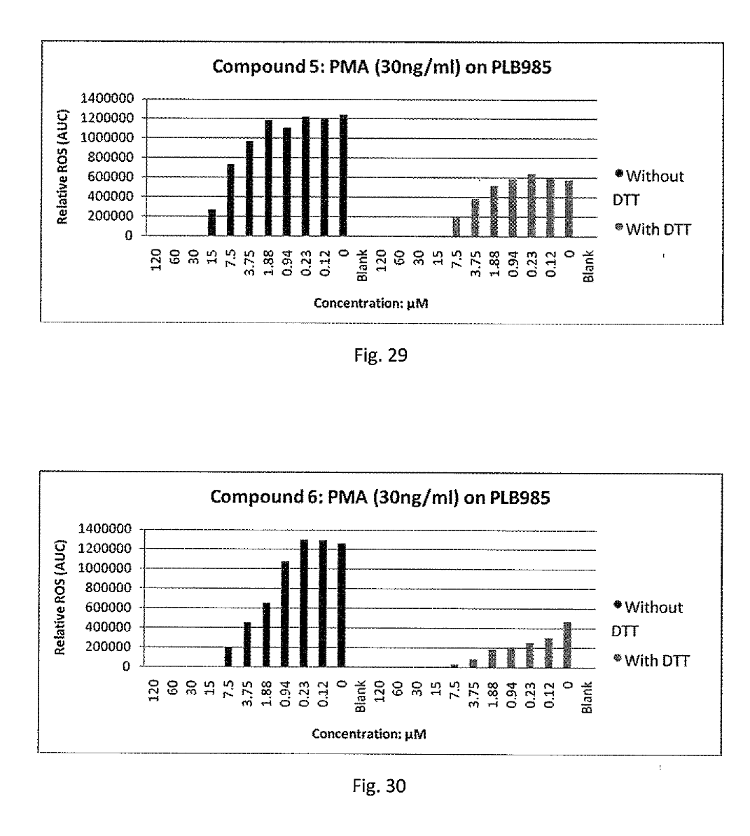

FIG. 29: Extracellular ROS analyzed by Isoluminol induced luminescence in human PLB-985 cell line. ROS induced with PMA (30 ng/L). Concentrations of compound 5: 120; 60; 30; 15; 7.5; 3.75; 1.88; 0.94; 0.23; 0.12; and 0 microM. Left panel: Without DTT. Right panel: With DTT 100 microM.

FIG. 30: Extracellular ROS analyzed by Isoluminol induced luminescence in human PLB-985 cell line. ROS induced with PMA (30 ng/L). Concentrations of compound 6: 120; 60; 30; 15; 7.5; 3.75; 1.88; 0.94; 0.23; 0.12; and 0 microM. Left panel: Without DTT. Right panel: With DTT 100 microM.

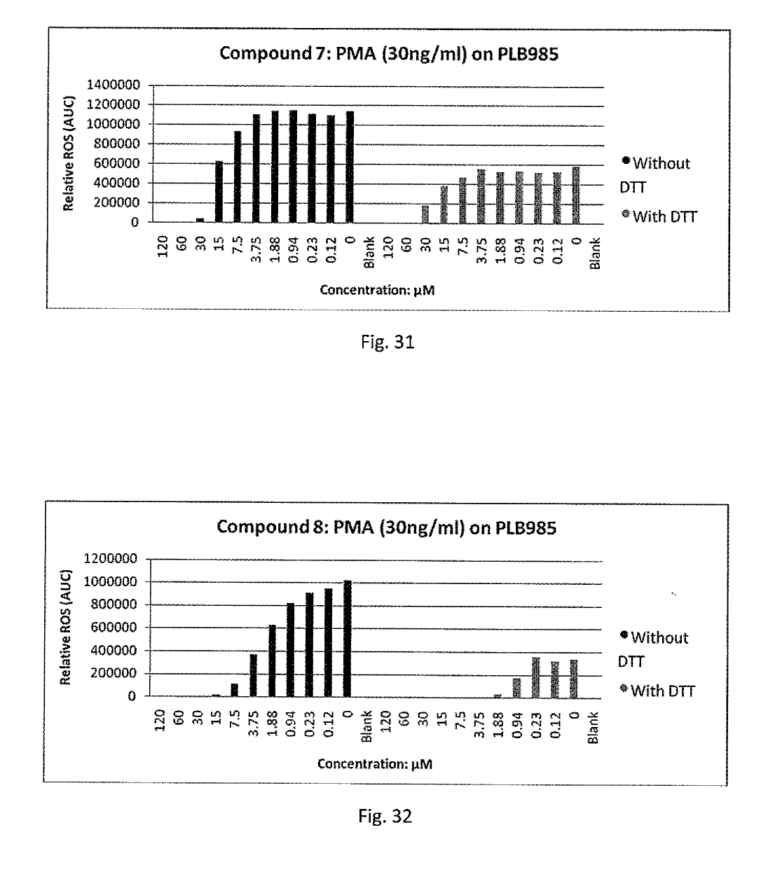

FIG. 31: Extracellular ROS analyzed by Isoluminol induced luminescence in human PLB-985 cell line. ROS induced with PMA (30 ng/L). Concentrations of compound 7: 120; 60; 30; 15; 7.5; 3.75; 1.88; 0.94; 0.23; 0.12; and 0 microM. Left panel: Without DTT. Right panel: With DTT 100 microM.

FIG. 32: Extracellular ROS analyzed by Isoluminol induced luminescence in human PLB-985 cell line. ROS induced with PMA (30 ng/L). Concentrations of compound 8: 120; 60; 30; 15; 7.5; 3.75; 1.88; 0.94; 0.23; 0.12; and 0 microM. Left panel: Without DTT. Right panel: With DTT 100 microM.

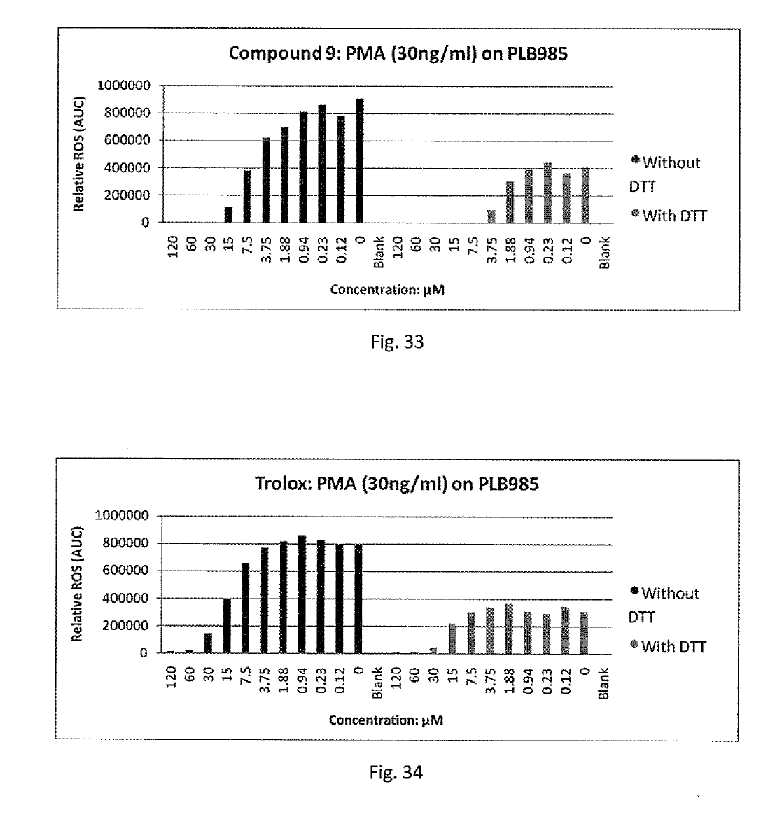

FIG. 33: Extracellular ROS analyzed by Isoluminol induced luminescence in human PLB-985 cell line. ROS induced with PMA (30 ng/L). Concentrations of compound 9: 120; 60; 30; 15; 7.5; 3.75; 1.88; 0.94; 0.23; 0.12; and 0 microM. Left panel: Without DTT. Right panel: With DTT 100 microM.

FIG. 34: Extracellular ROS analyzed by Isoluminol induced luminescence in human PLB-985 cell line. ROS induced with PMA (30 ng/L). Concentrations of Trolox: 120; 60; 30; 15; 7.5; 3.75; 1.88; 0.94; 0.23; 0.12; and 0 microM. Left panel: Without DTT. Right panel: With DTT 100 microM.

### Conclusions

From FIGS. 27 to 34 it can be seen that all tested compounds within the scope of the present invention show very good antioxidative properties. At high concentrations all tested substances of the present invention almost completely eradicate all ROS induced by PMA. The substances also show an excellent dose titration response. Addition of DTT further improves the antioxidative properties. However, DTT also decreases the ROS production from non-stimulated cells.

### Examples Series 7

### Organ Model. TBARS Experiment Showing the Effects of Compounds of the Present Invention in Preventing Oxidation of Tissue

The following protocol was followed:

All incubation are done in 37° C. heating cabinet
Add 3.9 ml 40 mM Hepes buffer pH 7.4 to a 50 ml tube and Argon bubble for 5 min.
Add 100 mg of kidney slice and incubate shaking with Argon bubbling for 5 min and thereafter shaking 15 min under argon
Add 0.1 ml of compound, vehicle or 400 μg desferal.
Bubble 1 min with Argon and thereafter 20 minutes shaking under Argon
Mince the tissue with a scissor with a subsequent 10 minute shaking under Argon
Re-oxygenate with vigorously bubbling 95% oxygen for 1 minute and thereafter 30 minutes shaking
Add 100 μg of desferal in all tubes
Homogenize the tissue for 20 seconds under ice and snap freeze the sample on try ice.

### TBA-MDA Assay

Add 50 μl of homogenate to 500 μl 2% TCA, vortex and spin 12000×g for 2 minutes
Add 60 μl of supernatant to 110 μl 14 mM warm TBA solutions in a black 96 well plate
Incubate the plate at 80° C. in 40 minutes

Let the plate reach room temperature and measure the fluorescence using a plate reader (Ex 485 and Em 545)

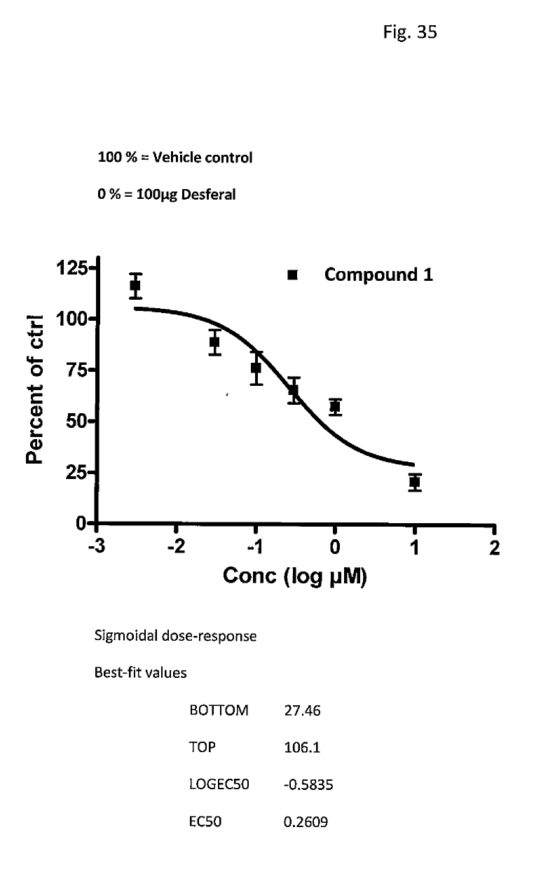

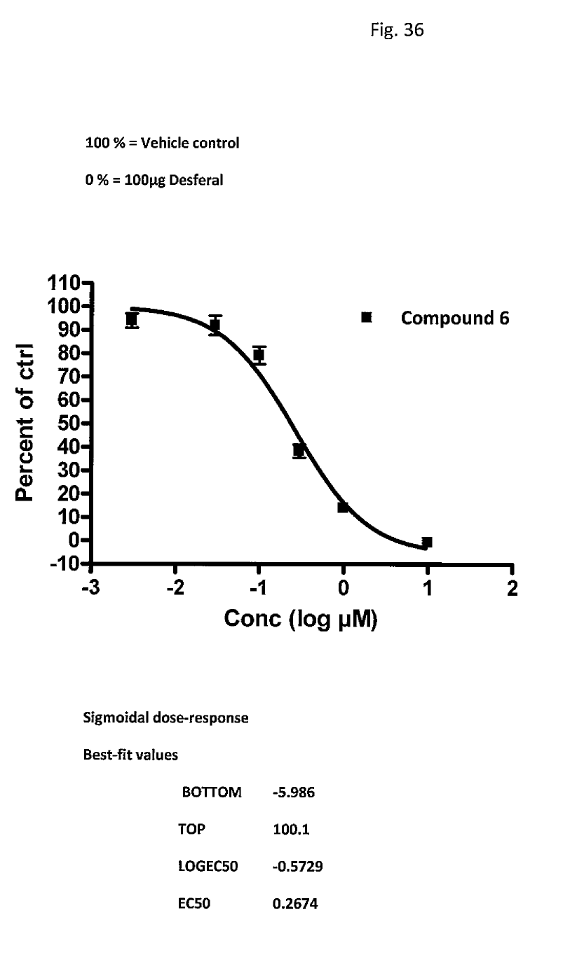

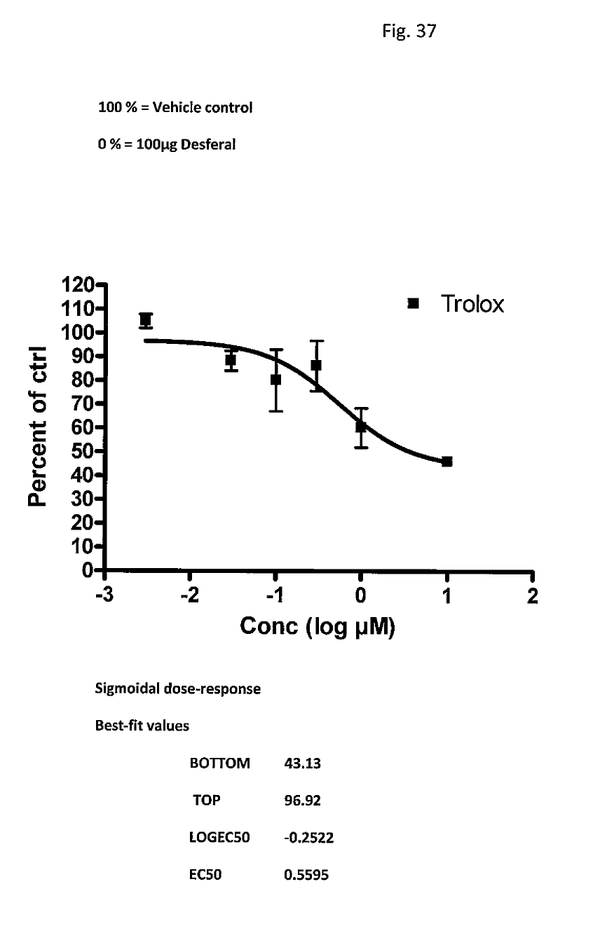

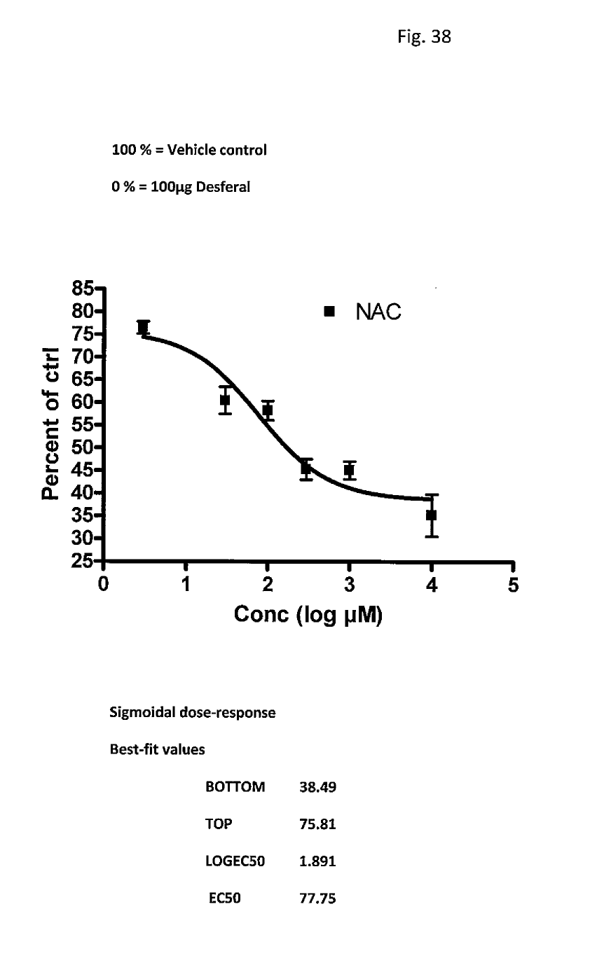

FIGS. 35 to 38 show the results of the TBARS experiments. FIGS. 35 to 38 show that sub-micromolar concentrations of compound 1 and compound 6 were able to inhibit 50% of the TBARS formation in the control incubation. Compounds 1 and 6 were more potent inhibitors of TBARS production than Trolox and N-acetylcysteine.

1. A method of antioxidant treatment of a subject in need thereof, comprising administering a therapeutically effective amount of a compound comprising formula I or formula II, or a pharmaceutically acceptable salt thereof: 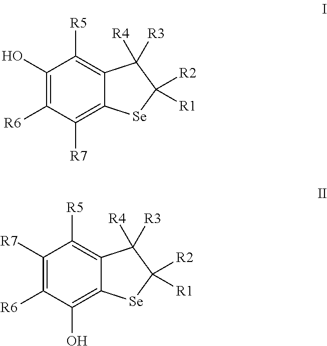
wherein each of R1, R2, R3 and R4 is independently selected from the group consisting of hydrogen, alkyl, and substituted alkyl; and
each of R5, R6 and R7 is independently selected from the group consisting of hydrogen, alkyl, substituted alkyl, alkoxy, substituted alkoxy, N,N-dialkylamino, substituted N,N-dialkylamino, N-monoalkylamino, substituted N-monoalkylamino, and electron-donating substituents.

2. The method of claim 1, wherein said compound comprises a compound of formula I, or a pharmaceutically acceptable salt thereof.

3. The method of claim 1, wherein said compound comprises a compound of formula II, or a pharmaceutically acceptable salt thereof.

4. The method of claim 1, wherein two of R1, R2, R3 and R4 are hydrogen and two are alkyl.

5. The method of claim 4, wherein each of said alkyls is independently selected from the group consisting of C₁₋₅ alkyl.

6. The method of claim 1, wherein three of R1, R2, R3 and R4 are hydrogen and one is alkyl.

7. The method of claim 6 wherein said alkyl is selected from the group consisting of C₁-C₅ alkyl.

8. The method of claim 1, wherein R5 is selected from the group consisting of hydrogen, C₁-C₅ alkyl, C₁-C₅ alkoxy, N,N-di(C₁-C₅)alkylamino and N-mono(C₁-C₅)alkylamino.

9. The method of claim 1, wherein R6 is selected from the group consisting of hydrogen, C₁-C₅ alkyl, C₁-C₅ alkoxy, N,N-di(C₁-C₅)alkylamino and N-mono(C₁-C₅)alkylamino.

10. The method of claim 1, wherein R7 is selected from the group consisting of hydrogen, C₁-C₅ alkyl, C₁-C₅ alkoxy, N,N-di(C₁-C₅)alkylamino and N-mono(C₁-C₅)alkylamino.

11. The method of claim 10, wherein R7 is hydrogen.

12. The method of claim 1, wherein one or both of R5 and R6 is not hydrogen.

13. The method of claim 1, wherein said compound acts as a catalytic antioxidant.

14. The method of claim 13, wherein said compound is administered in combination with a therapeutically effective amount of a pharmaceutically acceptable reducing agent.

15. The method of claim 14, wherein said reducing agent comprises a thiol.

16. The method of claim 14, wherein said reducing agent is selected from the group consisting of N-acetylcysteine, cysteine, dithiothreitol, glutathione, ascorbic acid and sodium ascorbate.

17. (canceled)

18. The method of claim 1, wherein said treatment is for a disorder or condition caused by or involving free radical-mediated tissue damage or oxidative tissue damage.

19. The method of claim 1, wherein said treatment is for a disorder or condition selected from the group consisting of ischemic injuries, reperfusion injuries, thrombosis, embolism, neoplasms, cancers, Parkinson's disease, Alzheimer's disease, atherosclerosis, allergic conditions, inflammatory conditions, bronchitis, asthma, rheumatoid arthritis, ulcerative cholitis, Crohn's disease, cataracts, respiratory distress syndrome, damage caused by chemicals, damage caused by radiation, damage caused by antineoplastic agents, damage caused by immunosuppressive agents, ischemia in the heart, ischemia in the kidney, ischemia in the CNS; reperfusion injury in the heart; reperfusion injury in the kidney; reperfusion injury in the CNS; post-operative ischemia; post-operative reperfusion injury; organ preservation, burn injury, wound healing, and IBS (irritable bowel syndrome).

20. The method of claim 19, wherein said disorder or condition is selected from the group consisting of organ preservation, burn injury, and rheumatoid arthritis.

21. (canceled)

22. (canceled)

23. (canceled)

24. (canceled)

25. (canceled)

26. (canceled)

27. (canceled)

28. A pharmaceutical composition comprising:
(a) a compound comprising formula I or formula II, or a pharmaceutically acceptable salt thereof: 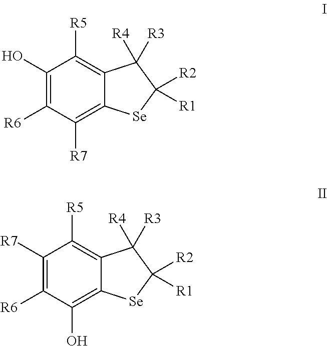
wherein each of R1, R2, R3 and R4 is independently selected from the group consisting of hydrogen, alkyl, and substituted alkyl; and
each of R5, R6 and R7 is independently selected from the group consisting of hydrogen, alkyl, substituted alkyl, alkoxy, substituted alkoxy, N,N-dialkylamino, substituted N,N-dialkylamino, N-monoalkylamino, substituted N-monoalkylamino, and electron-donating substituents;
and
(b) a pharmaceutically acceptable diluent, excipient or carrier.

29. The pharmaceutical composition of claim 28, further comprising:
(c) a pharmaceutically acceptable reducing agent.
